SEQUENCE LISTING (16 sequences)

SEQ ID NO: 1 - protein, 449 aa, Artificial Sequence
  artificial antibody heavy chain sequence
EVKFEESGGGLVQPGGSMKLSCVASGFSFSNYWMNWVRQSPEKGLEWVAEIRLTSNKQAIYYAESVKGRFTISRDDSKSSVYLQMNNLRAEDTGIYYCASLFYDGYLHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 218 aa, Artificial Sequence
  artificial antibody light chain sequence
DIVLTQSPASLAVSLGQRATISCRASESVGNFGISFMNWFQQKPGQPPKLLIYTASNQGSGVPARFSGSGSGTDFSLNIHPMEEDDSAMYFCQQSKEIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 450 aa, ARTIFICIAL SEQUENCE
  artificial antibody heavy chain sequence
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 217 aa, Artificial Sequence
  artificial antibody light chain sequence
AIQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 578 aa, Artificial Sequence
  artificial antibody heavy chain sequence
EVKFEESGGGLVQPGGSMKLSCVASGFSFSNYWMNWVRQSPEKGLEWVAEIRLTSNKQAIYYAESVKGRFTISRDDSKSSVYLQMNNLRAEDTGIYYCASLFYDGYLHWGQGTLVTVSSPAPNLLGGPEVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 338 aa, Artificial Sequence
  artificial antibody light chain sequence
DIVLTQSPASLAVSLGQRATISCRASESVGNFGISFMNWFQQKPGQPPKLLIYTASNQGSGVPARFSGSGSGTDFSLNIHPMEEDDSAMYFCQQSKEIPWTFGGGTKLEIKSPAPNLLGGPAIQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 578 aa, Artificial Sequence; identical to SEQ ID NO: 5
  artificial antibody heavy chain sequence

SEQ ID NO: 8 - protein, 334 aa, Artificial Sequence
  artificial antibody light chain sequence
DTQMTQSPSSLSASVGDRVTITCRASQSISSYLAWYQQKPGKAPKLLIYAASSLYSGVPSRFSGSGSGTDFTLTISSLQPEDFATYYCQQYSSLPYTFGQGTKLEIKSPAPNLLGGPAIQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 701 aa, Artificial Sequence
  artificial antibody heavy chain sequence
EVKFEESGGGLVQPGGSMKLSCVASGFSFSNYWMNWVRQSPEKGLEWVAEIRLTSNKQAIYYAESVKGRFTISRDDSKSSVYLQMNNLRAEDTGIYYCASLFYDGYLHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGSASGGSEVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVSSGGGGSGGGGSGGGGSAIQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIK

SEQ ID NO: 10 - protein, 218 aa, Artificial Sequence; identical to SEQ ID NO: 2
  artificial antibody light chain sequence

SEQ ID NO: 11 - protein, 702 aa, Artificial Sequence [sequence omitted]

SEQ ID NO: 12 - protein, 218 aa, Artificial Sequence; identical to SEQ ID NO: 2
  artificial antibody light chain sequence

SEQ ID NO: 13 - protein, 698 aa, Artificial Sequence
  artificial antibody heavy chain sequence
EVQLVESGGGLVQPGGSLRLSCAASGFTFSSYAMSWIRQAPGKGLEWIGQISGSGGSTYYNDNVLGRFTISRDNSKNTLYLQMNSLRAEDTAVYYCARDSGYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGSASGGSEVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVSSGGGGSGGGGSGGGGSAIQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIK

SEQ ID NO: 14 - protein, 214 aa, Artificial Sequence
  artificial antibody light chain sequence
DTQMTQSPSSLSASVGDRVTITCRASQSISSYLAWYQQKPGKAPKLLIYAASSLYSGVPSRFSGSGSGTDFTLTISSLQPEDFATYYCQQYSSLPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 699 aa, Artificial Sequence
  artificial antibody heavy chain sequence
EVQLVESGGGLVQPGGSLRLSCAASGFTFSSYAMSWIRQAPGKGLEWIGQISGSGGSTYYNDNVLGRFTISRDNSKNTLYLQMNSLRAEDTAVYYCARDSGYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGSASGGSAIQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIKSGGGGSGGGGSGGGGSEVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVSS

SEQ ID NO: 16 - protein, 214 aa, Artificial Sequence; identical to SEQ ID NO: 14
  artificial antibody light chain sequence

(19) United States
(12) Patent Application Publication
(10) Pub. No.: US 2017/0253672 A1
(43) Pub. Date: Sep. 7, 2017

(54) REMOVAL OF MONOMERIC TARGETS

(71) Applicant: HOFFMANN-LA ROCHE INC., LITTLE FALLS, NJ (US)

(72) Inventors: Roland Beckmann, Vienna (AT); Kristian Jensen, Landshut (DE)

(21) Appl. No.: 15/456,325

(22) Filed: Mar. 10, 2017

Related U.S. Application Data
(62) Division of application No. 14/123,041, filed on May 27, 2014, now abandoned.

(30) Foreign Application Priority Data
May 27, 2011 (EP) 11004372.6

(51) Int. Cl. C07K 16/46; C07K 16/24

(52) U.S. Cl. CPC C07K 16/468; C07K 16/248; C07K 2317/21; C07K 2317/31; C07K 2317/33; C07K 2317/622; C07K 2317/76; C07K 2317/92; C07K 2317/55

(57) ABSTRACT

The present invention relates to a novel method for the removal of monomeric targets from bodily fluids, and to pharmaceutical compositions for use in such methods.

### CROSS-REFERENCE TO RELATED APPLICATIONS

The present invention is a divisional of U.S. patent application Ser. No. 14/123,041, having a 35 U.S.C. §371(c) date of May 27, 2014, which is the U.S. national phase of International Application No. PCT/EP2012/002280, filed May 29, 2012, which designated the U.S. and claims priority to European patent application No. 11004372.6, filed May 27, 2011, each of which is hereby incorporated by reference in its entirety.

### SEQUENCE LISTING

The instant application contains a Sequence Listing which has been submitted in ASCII format via EFS-Web and is hereby incorporated by reference in its entirety. Said ASCII copy, created on Mar. 9, 2017, is named WR_PAT_DUT_3USDIV1_SeqListing.txt and is 60 kilobytes in size.

### FIELD OF THE INVENTION

The present invention relates to a novel method for the removal of monomeric targets from bodily fluids, and to pharmaceutical compositions for use in such methods.

### BACKGROUND OF THE INVENTION

This invention relates to a novel method for the removal of monomeric targets from bodily fluids.

Recombinant antibodies are in use as active ingredients in a wide variety of drugs approved for clinical use. An important group of potential antibody drug targets are monomeric soluble proteins contributing to disease, including many monomeric cytokines and chemokines. This category of targets is highly significant and problematic as it comprises many molecules implicated in human diseases, but antibodies directed against them have not shown the impressive efficacy seen with some targets in other classes of target molecules, and none have received marketing authorization to date. The problem that is common to all of the monomeric targets is the unresolved question of how to reliably achieve good pharmacokinetics of any antibody directed against them. In the case of multimeric soluble targets, bivalent IgG-type antibodies form immune complexes, which vary in size depending on target, epitope, target concentration and antibody concentration. These immune complexes are efficiently cleared by the mononuclear phagocyte system (MPS, also referred to as reticuloendothelial system, RES) and/or by adhesion to red cells via CR1 receptors and subsequent shedding in the spleen or liver, thus lowering the concentration of soluble multimeric target in the patient. In contrast, current antibodies directed against monomers cannot form larger immune complexes and rather than efficiently clearing these targets can merely bind them and remain in circulation as tiny, long-lived singular complexes comprising one antibody and one to two target molecules. Since the monomers can dissociate from the antibody and re-associate, the antibody can dramatically increase the in vivo concentration of bioavailable pathogenic target molecules. It has long been known that for this reason, anti-cytokine antibodies can enhance and prolong the in vivo effects of cytokines such as IL-3, IL-4 and IL-7 in mice (Finkelmann et al., J Immunol. 1993 Aug. 1; 151(3):1235-44). Fewer data are available for treatment of humans, but a well-known example is that of anti-IL-6 clinical studies. Therapeutic antibodies against the monomer IL-6 have resulted in a dramatic, up to 1,000-fold increase of IL-6 serum concentrations in patients, rather than a reduction (Lu et al., Blood. 1995 Oct. 15; 86(8):3123-31; Klein & Brailly, Immunol Today 1995; 16:216-220, Rossi et al., Bone Marrow Transplant. 2005 November; 36(9):771-9). The dramatic increase in serum levels of IL-6 following the treatment with anti-IL-6 antibody was associated with the fact that the serum half-life of IL-6 was increased 200-fold in patients due to the administration of anti-IL-6 antibody (Lu et al., Blood. 1995 Oct. 15; 86(8):3123-31). Most of the increased soluble IL-6 in patients treated with anti-IL-6 antibody was bound to the therapeutic antibody (Rossi et al, loc. cit.). The singular immune complexes comprising 1 antibody molecule and 1-2 IL-6 molecules thereby in effect form a pool of IL-6 with a long half-life that can be released as free pathogenic IL-6 by dissociation from the antibody. This makes anti-IL-6 therapy with a monoclonal antibody drug a serious problem for which there is no obvious solution. Importantly, the good phase III clinical results that were obtained when the IL-6 receptor was targeted in a different indication using the antibody toclizumab (Burmester et al., Ann Rheum Dis. 2011 May; 70(5):755-9. Epub 2010 Dec. 27.) demonstrates that interference with the IL-6 pathway as such is not a particular problem unique to this pathway, but rather confirms that it is the technology of current monospecific monoclonal antibodies directed against monomeric targets that is inadequate.

In contrast to soluble monomeric targets, there are well-validated multimeric soluble targets against which approved, currently marketed antibody drugs are directed. These soluble multimeric proteins that are currently successfully being treated in human diseases with approved antibody drugs include TNF-alpha, treated with antibodies adalimumab and infliximab, and VEGF165, treated with approved antibody bevacizumab. A common feature of these successful antibodies directed against soluble multimeric targets is that they have the potential to form multimeric immune complexes with the soluble multimeric targets, thereby resulting in their clearance through the mononuclear phagocyte system MPS (Tabrizi et al., Drug Discov Today. 2006 January; 11(1-2):81-8). TNF-alpha is a soluble trimeric protein, with a typical TNF-alpha molecule comprising three identical copies of the TNF-alpha polypeptide and having multiple copies of the epitopes recognised by antibodies adalimumab and infliximab, respectively. This allows the formation of immune complexes between the anti-TNF-alpha drugs and the TNF-alpha trimer. The potential size of the immune complexes between TNF-alpha (52 kDa) and adalimumab (150 kDa) or infliximab (average 165 kDa) has been investigated by Amgen-based authors Khono et al., using size exclusion chromatography-light scattering assays. Adalimumab and infliximab formed a variety of complexes with TNF with molecular weights as high as 4,000 and 14,000 kDa, respectively, suggesting the presence of complexes with a wide range of sizes and stoichiometries. The anti-TNF antibodies also formed visible lines of precipitation in Ouchterlony assays.

The authors also tested Etanercept, a different approved TNF-alpha antagonist that is a soluble TNF receptor-Fc fusion protein. Etanercept did not form large complexes with TNF-alpha but rather two types of complexes of 180 and 300 kDa, representing one and two etanercept monomers bound to a TNF trimer, respectively. Interestingly, in an animal model of RA driven by a human TNF transgene (Kaymakcalan et al., Arthritis Rheum. 46 (Suppl.) (2002) S304), TNF alpha was cleared more slowly from serum following administration of Etanercept than after adalimumab or infliuximab, suggesting that the small, non-aggregated TNF-Etanercept complexes persisted longer in the serum. In the same animal model, Etanercept was also less effective than adalimumab. In humans, Etanercept is also an efficacious anti-TNF-alpha drug in the treatment of RA, but it is very important to note that the pharmacokinetics of Etanercept cannot be compared to the antibody drugs, as it has a much shorter half-life of only 3.5 to 5 days in patients compared to 10-20 days for adalimumab and approximately 9.5 days for infliximab. Therefore, Etanercept cannot produce a build-up of TNF-alpha concentrations in the serum to the same extent as a non-aggregating antibody drug with a long half-life would do. Nonetheless, it is interesting that Etanercept appears to be less efficacious than the antibody drugs in the treatment of Crohn's disease and psoriasis, although it is not known if the lack of aggregate formation by Etanercept is associated with this lesser efficacy (Scallon et al., Cytokine. 1995 November; 7(8):759-70M; Van den Brande et al., Gastroenterology. 2003 June; 124(7):1774-85).

Human VEGF165 is also a soluble multimeric protein, being a dimer that comprises two identical polypeptides. The approved anti-VEGF antibody drug bevacizumab has the potential to aggregate the dimeric VEGF protein, as illustrated by the crystal structure of the VEGF-bevacizumab Fab complex (Structure 1 BJ1; Muller et al., Structure (1998) 6 p. 1153-1167). In the complex, the bevacizumab Fab binds to an epitope on VEGF of which two highly exposed copies exist at opposite poles of each dimeric VEGF molecule. The bevacizumab-VEGF aggregates are predicted to be predominantly heterotrimeric in patients, with each VEGF dimer being bound by two bevacizumab molecules. These immune complexes are efficiently cleared, with VEGF being permanently neutralized during the time between being bound by bevacizumab and being cleared. It should be noted that in patients treated with bevacizumab, a 3-fold to 4-fold rise in VEGF concentration above baseline is observed (Gordon et al., J Clin Oncol. 2001 February; 19(3): 843-50, Gordon et al., J Clin Oncol. 2001 February; 19(3): 851-6). This is probably as a result of the VEGF-bevacizumab complexes having a somewhat lower clearance rate compared to free VEGF (3.4-fold lower in rats; Hsei e al. Pharm Res. 2002 November; 19(11):1753-6). However, this rise is far less than what has been observed for monomeric targets (which can show dramatic 1000-fold increases in serum concentration as described above), cannot be compared with the problems observed with monomeric targets, and clinically is not problematic as the VEGF is permanently neutralized until it is cleared, as stated above.

Another multimeric soluble target protein is immunoglobulin E (IgE), which is also a dimer. The approved anti-IgE antibody drug omalizumab has proven efficacious for patients with asthma and allergic rhinitis. In vitro, omalizumab and human IgE form several immune complexes that vary in size as the two components' molar ratios are changed (Liu et al., Biochemistry, 1995, 34(33): 10474-82). The largest complex, a stable cyclical hexameric structure consisting of three IgE and three omalizumab molecules, is formed at a 1:1 molar ratio. With excesses of either IgE or omalizumab, the distribution of complexes is dominated by a trimer consisting of one IgE and two omalizumab molecules or vice versa. The IgE-omalizumab immune complexes are efficiently cleared, with IgE being permanently neutralized during the time between being bound by omalizumab and being cleared. The total serum-level of IgE in treated patients is increased up to 5-fold (Chang, Nat. Biotechnol., 2000, 18(2): 157-62; Milgrom et al., N Engl J Med., 1999, 341(26): 1966-73). However, once again compared to the singular immune complexes formed between antibody drugs and monomeric targets, the increase in serum-level of the multimeric target is minimal and has no adverse effect due to the neutralization of IgE in its omalizumab-bound state.

However, despite that fact that many attempts have been made to address the issue of efficient and safe removal of soluble monomeric targets from bodily fluids, so far these attempts have had limited success.

Interestingly, Montero-Julian et al. (Blood. 1995 Feb. 15; 85(4):917-24) performed a pharmacokinetic study in mice injected with radiolabeled IL-6 and various anti-IL-6 monoclonal antibodies. The elimination of radiolabeled IL-6 was rapid in untreated animals with a mean residence times of IL-6 in the central compartment of 70 min and the label rapidly appearing in the kidneys. Clearance was much slower (but not increased as greatly as in patients) with a mean residence time of 600 min when mice were treated with one anti-IL-6 antibody, and the label remained in the serum. Importantly, in mice treated with a combination of three antibodies directed against different epitopes, IL-6 was cleared rapidly with a mean residence times of IL-6 of 70 min and possibly as low as 5 min in the central compartment, and the label appeared predominantly in the liver. These findings demonstrated that IL-6 can potentially be cleared by being aggregated using a cocktail of several antibody molecules. However, the authors did not succeed in achieving a rapid clearance of IL-6 using only two antibodies. The reasons for this are not fully understood. However, a key factor would appear to be that the authors did not use any antibodies of murine IgG2a isotype which exhibits the highest complement fixing ability of the murine isotypes (Leatherbarrow and Dwek, Mol Immunol. 1984 April, 21(4): 321-7). Instead in one instance the authors used two antibodies of murine IgG1 isotype, which was historically believed to not activate the classical complement pathway at all and later discovered to bind complement only weakly and under certain conditions (Klaus et al., 1979, Immunology 38:687; Okada et al., Mol Immunol. 1983 Mar., 20(3): 279-85). In another instance the authors used one antibody of murine IgG1 isotype plus one antibody of murine IgG2b isotype which exhibits only intermediate complement binding affinity. Comparable experiments performed with two antibodies of murine IgG2a isotype would have been extremely interesting but have not been reported in the prior art. Besides the issue of having used unsuitable or less suitable isotypes, Montero-Julian et al. used antibodies of different affinities, and there may have been issues with their specific experimental set-up and possibly homogeneity of IL-6 that was used, since the authors also found that in the experiments with three antibodies, 25% to 30% of 1215-IL-6 was only in the form of lower molecular weight complexes corresponding to monomeric and dimeric immune complexes, and that maximal binding of 1125-IL-6 to each of the three MoAbs was only 75% to 85% in a liquid-phase assay, suggesting that the population of IL-6 molecules may not have been homogenous. Based on their findings, the authors suggested that the use of a cocktail of three antibodies, binding simultaneously to a cytokine, provides a new means of enhancing the clearance of the target molecule.

While treating patients with cocktails of monoclonal antibodies is an interesting proposal, there are serious medical and economic issues with such an approach. Medically, using a cocktail of antibodies would mean that each component of the drug may have different pharmacokinetic behaviour, thereby changing the composition of the cocktail during the treatment. Furthermore, cocktails of recombinant proteins bear the risk of being more immunogenic than a single drug molecule. Economically, the independent parallel development of several recombinant monoclonal antibody drugs would be extremely costly and may not be a viable option.

Thus, there remained still a large unmet need to develop a method that uses a single active ingredient to remove such monomeric target biomolecules from a bodily fluid rapidly, efficiently and without the existence of freely circulating complexes comprising a binding molecule and bound target biomolecule(s), which can be an undesired source for the liberation of the target biomolecule and thus lead to an undesired increase of the available biomolecule concentration in the bodily fluid.

The solution for this problem that has been provided by the present invention, i.e. the use of an antibody molecule having at least two different binding specificities, has so far not been achieved or suggested by the prior art.

### SUMMARY OF THE INVENTION

The present invention relates to a novel method for the removal of soluble monomeric biomolecules from bodily fluids by using binding molecules with at least two different specificities, either for two different epitopes on the monomeric biomolecule, or for one epitope on the biomolecule and a second epitope on a second biomolecule, that exhibits at least two copies of the second epitope. By contacting the bodily fluid containing the soluble monomeric biomolecule, or alternatively the soluble monomeric biomolecule and the second biomolecule with the binding molecule, aggregates are being formed that result in the removal of the soluble monomeric biomolecule from the bodily fluid.

Thus, in a first aspect, the present invention relates to a method for removing a soluble monomeric biomolecule from a bodily fluid by the formation of multimeric complexes using a binding molecule comprising at least two different binding sites, wherein at least one binding site is specific for an epitope present on said biomolecule, comprising the step of: contacting said bodily fluid with said bispecific binding molecule.

In a second aspect, the present invention relates to an antibody molecule comprising at least two independent paratopes, wherein the first paratope can specifically bind a first epitope of a soluble monomeric biomolecule and the second paratope can specifically bind a different second epitope on said monomeric biomolecule.

In a third aspect, the present invention relates to an antibody molecule comprising at least two independent paratopes, wherein the first paratope is able to specifically bind a first epitope present on monomeric soluble target molecule and the second paratope is able to specifically bind a second epitope present on a multimeric soluble target molecule.

In particular embodiments, both paratopes of said binding molecule bind to their respective epitopes on said soluble monomeric biomolecule in a way, which inhibit binding of said epitopes to their native binding partners required for signalling.

In a fourth aspect, the present invention relates to a pharmaceutical composition comprising the antibody molecule of the present invention, and optionally a pharmaceutically acceptable carrier and/or excipient.

In a fifth aspect, the present invention relates to a binding molecule comprising at least two different binding sites, wherein at least one binding site is specific for an epitope present on a soluble monomeric target biomolecule, for use in removing said target biomolecule from a bodily fluid, wherein said removal occurs by the formation of multimeric complexes comprising said binding molecule and said target biomolecule.

In a sixth aspect, the present invention relates to a pharmaceutical composition comprising the binding molecule of the present invention, and optionally a pharmaceutically acceptable carrier and/or excipient.

### BRIEF DESCRIPTION OF THE DRAWINGS

FIG. 1 shows a schematic representation of the aggregation of a monomeric target by a bi-specific antibody directed against two epitopes on the soluble target biomolecule.

FIG. 2 shows a schematic representation of the aggregation of a monomeric target by a bi-specific antibody directed against the soluble monomeric target biomolecule and a soluble multimeric target.

FIGS. 3 A-D shows a demonstration of the co-binding of targets: FIG. 3A shows Fab5 immobilized and injection of 50 nM IL6 followed by 100 nM Fab4. FIG. 3B shows Fab4 immobilized and injection of 50 nM IL6 followed by 100 nM Fab4. FIG. 3C shows Fab5 immobilized and injection of 50 nM IL6 followed by 100 nM Fab5. FIG. 3D shows Fab4 immobilized and injection of 50 nM IL6 followed by 100 nM Fab.

FIG. 4 shows monospecific and bispecific antibodies against IL6.

FIGS. 5 A-F shows an analysis of immune complexes. FIG. 5A shows an analysis of antibody D-5H5L−IL6. FIG. 5B shows an analysis of antibody D-5H5L+IL6. FIG. 5C shows an analysis of antibody Mab4-5H5L−IL6. FIG. 5D shows an analysis of antibody Mab4-5H5L+IL6. FIG. 5E shows an analysis of antibody DVD-45−IL6. FIG. 5F shows an analysis of antibody DVD-45+IL6.

FIG. 6 shows target-dependent C1q binding.

FIGS. 7 A-B shows the potency of a bivalent construct in a cell-based assay. FIG. 7A shows the potency of a bivalent construct in a 7TD1 cell-based assay. FIG. 7B shows the potency of a bivalent construct in a B9 cell-based assay.

FIG. 8 shows a graphical depiction of the PKPD model used.

FIGS. 9 A-B show PKPD modeling results. FIG. 9A shows results of using model A. FIG. 9B shows the results of using model B (The concentration of free cytokine is the third curve from the bottom). The y-scale is concentration in molar. The x-scale is time in days.

FIGS. 10 A-D shows results of the PKPD model: FIG. 10A shows a concentration curve of free target according to model A without antibody. FIG. 10B shows a concentration curve of free target according to model B without antibody. FIG. 10C shows a concentration curve of free target according to model A with antibody. FIG. 10D shows a concentration curve of free target according to model B with antibody. The y-scale is concentration in molar. The x-scale is time in seconds.

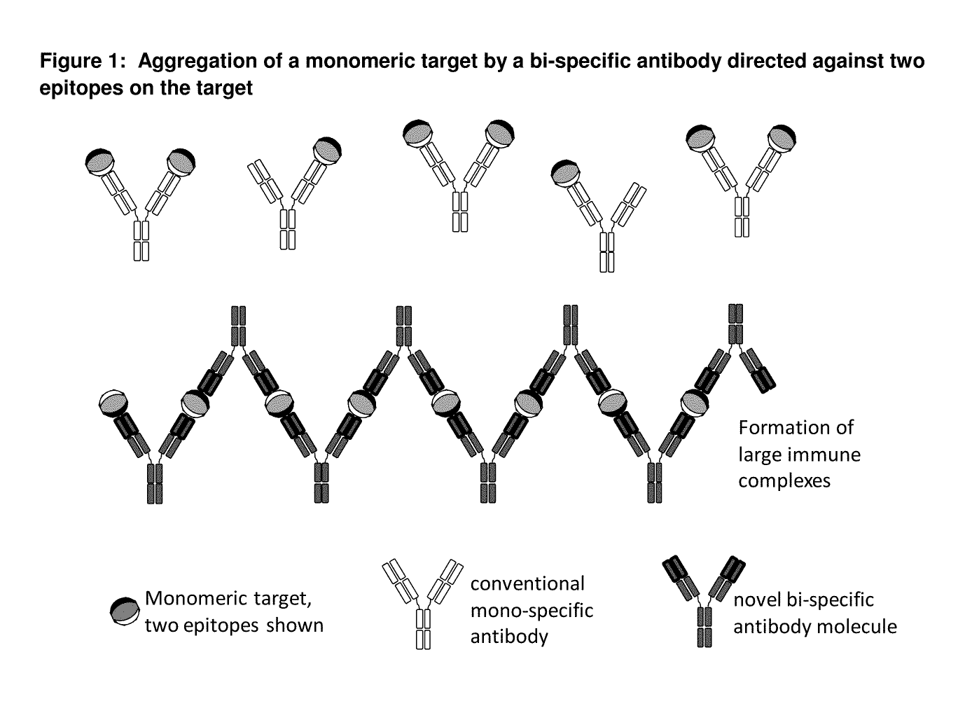

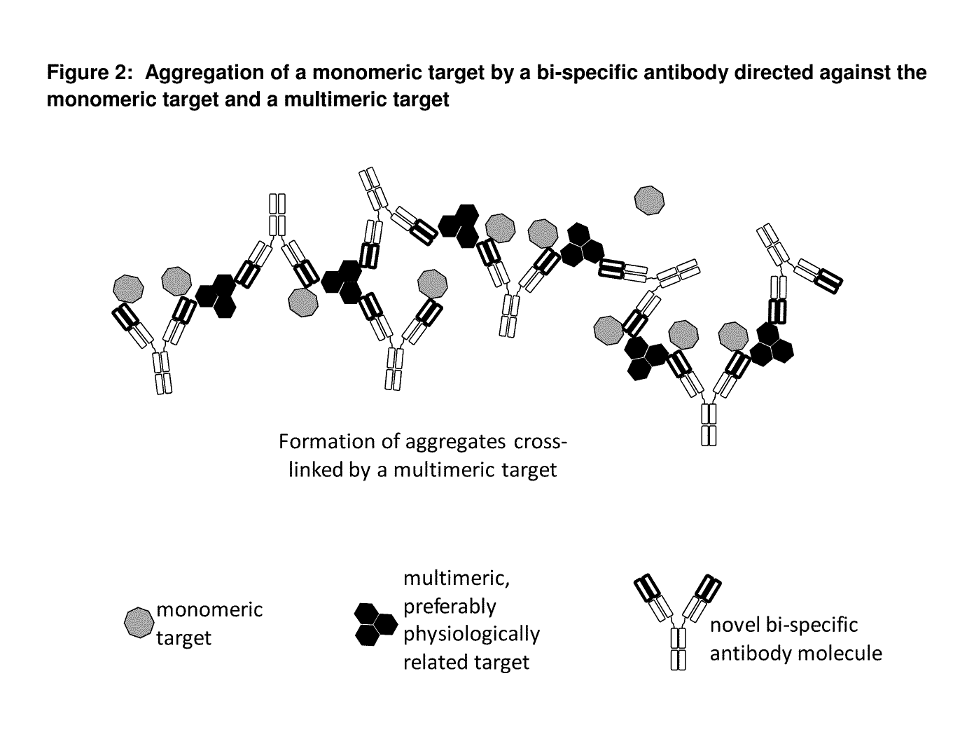

### DETAILED DESCRIPTION OF THE INVENTION

The peculiarity of this invention compared to former approaches for the removal of soluble monomeric biomolecules is the use of a binding molecule having at least two different binding sites, which results in the formation of molecular aggregates (large multi-valent immune complexes), whereas the prior art resulted in the formation of simple soluble antibody/antigen complexes, also known as singular immune complexes.

Thus, the present invention relates to a method for removing a soluble monomeric biomolecule from a bodily fluid by the formation of multimeric complexes using a binding molecule comprising at least two different binding sites, wherein at least one binding site is specific for an epitope present on said biomolecule, comprising the step of: contacting said bodily fluid with said bispecific binding molecule.

In the context of the present invention, the term “soluble . . . biomolecule” refers to a biomolecule that is present in the bodily fluid in free form, i.e. not anchored to a cell or tissue. A soluble biomolecule may be present as a homogeneous single molecule, or as a heterogeneous complex of two or more molecules, provided that each of the epitopes required for reaction with the binding molecule of the present invention is accessible.

The term “biomolecule” refers to any molecule that may be present in the bodily fluid, including peptides, proteins, glycopeptides and glycoproteins, phosphorylated peptides and proteins, sugars, nucleic acid sequences, and other organic compounds.

In the context of the present invention, the term “monomeric biomolecule” refers to a biomolecule that presents a given epitope only once per molecule. Thus, the term includes both single molecules and heterodimers presenting only one copy of a given epitope, whereas a single molecule having, for example, a repeat unit with an epitope appearing two or more times in the single molecule is not within the scope of the definition.

As used herein, a binding molecule is “specific to/for”, “specifically recognizes”, or “specifically binds to” a target, such as a target biomolecule (or an epitope of such biomolecule), when such binding molecule is able to discriminate between such target biomolecule and one or more reference molecule(s), since binding specificity is not an absolute, but a relative property. In its most general form (and when no defined reference is mentioned), “specific binding” is referring to the ability of the binding molecule to discriminate between the target biomolecule of interest and an unrelated biomolecule, as determined, for example, in accordance with a specificity assay methods known in the art. Such methods comprise, but are not limited to Western blots, ELISA, RIA, ECL, IRMA tests and peptide scans. For example, a standard ELISA assay can be carried out. The scoring may be carried out by standard colour development (e.g. secondary antibody with horseradish peroxide and tetramethyl benzidine with hydrogen peroxide). The reaction in certain wells is scored by the optical density, for example, at 450 nm. Typical background (=negative reaction) may be about 0.1 OD; typical positive reaction may be about 1 OD. This means the ratio between a positive and a negative score can be 10-fold or higher. Typically, determination of binding specificity is performed by using not a single reference biomolecule, but a set of about three to five unrelated biomolecules, such as milk powder, BSA, transferrin or the like.

In the context of the present invention, the term “about” or “approximately” means between 90% and 110% of a given value or range.

However, “specific binding” also may refer to the ability of a binding molecule to discriminate between the target biomolecule and one or more closely related biomolecule(s), which are used as reference points. Additionally, “specific binding” may relate to the ability of a binding molecule to discriminate between different parts of its target antigen, e.g. different domains, regions or epitopes of the target biomolecule, or between one or more key amino acid residues or stretches of amino acid residues of the target biomolecule.

In the context of the present invention, the term “epitope” refers to that part of a given target biomolecule that is required for specific binding between the target biomolecule and a binding molecule. An epitope may be continuous, i.e. formed by adjacent structural elements present in the target biomolecule, or discontinuous, i.e. formed by structural elements that are at different positions in the primary sequence of the target biomolecule, such as in the amino acid sequence of a protein as target, but in close proximity in the three-dimensional structure, which the target biomolecule adopts, such as in the bodily fluid.

In one embodiment, the binding molecule comprises at least a first and a second binding site with specificity for two different epitopes on said monomeric biomolecule.

In certain embodiments, said bispecific binding molecule comprises a first binding site with specificity for a first epitope on said soluble monomeric biomolecule, and a second binding site with specificity for a second epitope on a second soluble biomolecule present in said bodily fluid, wherein said second biomolecule comprises at least two copies of said second epitope.

In particular embodiments, the binding molecule is a bispecific molecule, particularly a bispecific antibody molecule.

As used herein, the term “antibody molecule” refers to an immunoglobulin (Ig) molecule that is defined as a protein belonging to the class IgG, IgM, IgE, IgA, or IgD (or any subclass thereof), which includes all conventionally known antibodies and functional fragments thereof. A “functional fragment” of an antibody/immunoglobulin molecule hereby is defined as a fragment of an antibody/immunoglobulin molecule (e.g., a variable region of an IgG) that retains the antigen-binding region. An “antigen-binding region” of an antibody typically is found in one or more hypervariable region(s) (or complementarity-determining region, “CDR”) of an antibody molecule, i.e. the CDR-1, -2, and/or -3 regions; however, the variable “framework” regions can also play an important role in antigen binding, such as by providing a scaffold for the CDRs. Preferably, the “antigen-binding region” comprises at least amino acid residues 4 to 103 of the variable light (VL) chain and 5 to 109 of the variable heavy (VH) chain, more preferably amino acid residues 3 to 107 of VL and 4 to 111 of VH, and particularly preferred are the complete VL and VH chains (amino acid positions 1 to 109 of VL and 1 to 113 of VH; numbering according to WO 97/08320). A preferred class of antibody molecules for use in the present invention is IgG. “Functional fragments” of the invention include the domain of a F(ab′)2 fragment, a Fab fragment, scFv or constructs comprising single immunoglobulin variable domains or single domain antibody polypeptides, e.g. single heavy chain variable domains or single light chain variable domains. The F(ab′)2 or Fab may be engineered to minimize or completely remove the intermolecular disulphide interactions that occur between the CH1 and CL domains.

An antibody with binding specificity for the target biomolecule of the second biomolecule, or for an epitope in the target biomolecule or second biomolecule, may be derived from immunizing an animal, or from a recombinant antibody library, including an antibody library that is based on amino acid sequences that have been designed in silico and encoded by nucleic acids that are synthetically created. In silico design of an antibody sequence is achieved, for example, by analyzing a database of human sequences and devising a polypeptide sequence utilizing the data obtained therefrom. Methods for designing and obtaining in silico-created sequences are described, for example, in Knappik et al., J. Mol. Biol. (2000) 296:57; Krebs et al., J. Immunol. Methods. (2001) 254:67; and U.S. Pat. No. 6,300,064 issued to Knappik et al.

In the context of the present invention, the term “bispecific antibody molecule” refers to an antibody molecule, including a functional fragment of an antibody molecule, that comprises specific binding sites for two different targets biomolecules, or two different epitopes, either present on one target biomolecule, or present on two different molecules, such as on the target biomolecule and a second biomolecule.

Bispecific antibody molecules may be obtained or prepared by a variety of different approaches.

In a first approach, the two paratopes recognizing two targets or epitopes do not both lie within one heterodimeric antibody variable region formed by one complementary VH-VL pair and do not both comprise CDR residues belonging to the same complementary VH-VL pair, so that at least two variable regions with different binding specificities are present. Numerous and diverse examples of such bispecific antibodies have been described, incl. diabodies (Perisic et al., Structure. 1994 Dec. 15; 2(12):1217-26; Kontermann, Acta Pharmacol Sin. 2005 January; 26(1):1-9; Kontermann, Curr Opin Mol Ther. 2010 April; 12(2):176-83.), TandAbs (Cochlovius et al., Cancer Res. 2000 Aug. 15; 60(16):4336-41.), single domains specific to different targets genetically fused by peptide linkers (e.g. Domantis: WO2008/096158; Ablynx: WO/2007/112940), or other constructs (for reviews, see: Enever et al., Curr Opin Biotechnol. 2009 August; 20(4):405-11. Epub 2009 Aug. 24.; Carter, Nat. Rev. Immunol. 6, 343 (2006); P. Kufer et al., Trends Biotechnol. 22, 238 (2004)).

In a second approach, bispecific antibodies comprise an IgG-like molecule and one or several additional appended binding domains or entities. Such antibodies include IgG-scFv fusion proteins in which a single chain Fv has been fused to one of the termini of the heavy chains or light chains (Coloma and Morrison, Nat. Biotechnol. 1997 February; 15(2):159-63), and dual variable domain (dvd-IgG) molecules in which an additional VH domain and a linker are fused to the N-terminus of the heavy chain and an additional VL domain and a linker are fused to the N-terminus of the light chain (Wu et al., Nat. Biotechnol. 2007 November; 25(11):1290-7).

In a third approach, bispecific antibodies comprise IgG-like antibodies that have been generated or modified in such a way that they exhibit two specificities without the addition of a further binding domain or entity. Such antibodies include IgG molecules, in which the naturally homodimeric CH3 domain has been modified to become heterodimeric, e.g. using an engineered protuberation (Ridgway et al., Protein Eng. 1996 July; 9(7):617-21), using strand exchange (Davis et al., Protein Eng Des Sel. 2010 April; 23(4):195-202. Epub 2010 Feb. 4), or using engineered opposite charges (Novo Nordisk), thereby potentially enabling the two halves of the IgG-like molecule to bind two different targets through the binding entities added to the Fc region, usually N-terminal Fab regions. Antibodies in this third group of examples also include IgG molecules in which some structural loops not naturally involved in antigen contacts are modified to bind a further target in addition to one bound naturally through variable region CDR loops, for example by point mutations in the Fc region (e.g. Xencor Fcs binding to FcgRIIb) or by diversification of structural loops.

In a fourth approach, the bispecific antibodies have two paratopes specific for two targets, where the two paratopes both comprise CDR residues located within the same heterodimeric VH-VL antibody variable region.

First, cross-reactive antibodies may be used, which have a single broad specificity that corresponds to two or more structurally related antigens or epitopes. For such antibodies the two antigens have to be related in sequence and structure. For example, antibodies may cross-react with related targets from different species, such as hen egg white lysozyme and turkey lysozyme (WO 92/01047) or with the same target in different states or formats, such as hapten and hapten conjugated to carrier (Griffiths A D et al. EMBO J 1994 13: 14 3245-60). It is possible to deliberately engineer antibodies for cross-reactivity. For example, antibodies have been engineered to recognise two related antigens from different species (example Genentech: antibody binding human LFA1 engineered to also bind rhesus LFA1, resulting in successful drug Raptiva/Efalizumab). Similarly, WO 02/02773 describes antibody molecules with “dual specificity”. The antibody molecules referred to are antibodies raised or selected against multiple structurally related antigens, with a single binding specificity that can accommodate two or more structurally related targets.

Second, there are polyreactive autoantibodies which occur naturally (Casali & Notkins, Ann. Rev. Immunol. 7, 515-531). These polyreactive antibodies have the ability to recognise at least two (usually more) different antigens or epitopes that are not structurally related. It has also been shown that selections of random peptide repertoires using phage display technology on a monoclonal antibody will identify a range of peptide sequences that fit the antigen binding site. Some of the sequences are highly related, fitting a consensus sequence, whereas others are very different and have been termed mimotopes (Lane & Stephen, Current Opinion in Immunology, 1993, 5, 268-271). It is therefore clear that the binding sites of some heterodimeric VH-VL antibodies have the potential to bind to different and sometimes unrelated antigens.

A third method described in the art that allows the deliberate engineering of bi-specific antibodies able to bind two structurally unrelated targets through two paratopes, both residing within one complementary heterodimeric VH-VL pair and both comprising CDR residues belonging to this complementary VH-VL pair, relates to “two-in-one” antibodies. These “two-in-one” antibodies are engineered to comprise two overlapping paratopes using methods somewhat distinct from previous cross-reactivity-engineering methods. This work has been described in WO 2008/027236 and by Bostrom et al. (Bostrom et al., Science. 2009 Mar. 20; 323(5921):1610-4.). In the published examples, a heterodimeric VH-VL antibody variable region specific for one target (HER2) was isolated and thereafter the light chain was re-diversified to achieve additional specificity for a second target (VEGF or death receptor 5). For one of the resulting antibodies the binding was characterised by structure resolution and it was found that 11 out of 13 VH and VL CDR residues making contact with HER2 in one antibody-antigen complex also made contact with VEGF in the alternative antibody-antigen complex. While the published “two-in-one” antibodies retained nanomolar affinities for HER2, only one of the clones published by Bostrom et al. (2009) had a nanomolar affinity of 300 nM for the additional target, VEGF, while four other clones had micromolar affinities for the additional targets.

A fourth method described in the art that allows the deliberate engineering of bi-specific antibodies able to bind two structurally unrelated targets through two paratopes, both residing within one complementary heterodimeric VH-VL pair and both comprising CDR residues belonging to this complementary VH-VL pair, relates to antibodies comprising complementary pairs of single domain antibodies. WO 03/002609 and US 2007/026482 have described heterodimeric VH-VL antibodies, in which a heavy chain variable domain recognises one target and a light chain variable domain recognises a second structurally unrelated target, and in which the two single domains with different specificities are combined into one joint heterodimeric VH-VL variable region. In the published examples of such antibodies, the single domains were first separately selected as an unpaired VH domain or as an unpaired VL domain to bind the two unrelated targets, and afterwards combined into a joint heterodimeric VH-VL variable region specific to both targets

In another aspect, the present invention relates to an antibody molecule comprising at least two independent paratopes, wherein the first paratope can specifically bind a first epitope of a soluble monomeric biomolecule and the second paratope can specifically bind a different second epitope on said monomeric biomolecule.

In the context of the present invention, the term “paratope” refers to that part of a given antibody molecule that is required for specific binding between a target biomolecule and the antibody molecule. A paratope may be continuous, i.e. formed by adjacent amino acid residues present in the antibody molecule, or discontinuous, i.e. formed by amino acid residues that are at different positions in the primary sequence of the amino acid residues, such as in the amino acid sequence of the CDRs of the amino acid residues, but in close proximity in the three-dimensional structure, which the antibody molecule adopts.

In one embodiment, the first and second epitopes on said monomeric biomolecule do not overlap.

In the context of the present invention, the term “the first and second epitopes on said monomeric biomolecule do not overlap” refers to the situation that binding of the binding molecule to one of the epitopes is essentially independent of whether another binding molecule is already bound to the other epitope or not. The term “essentially independent” refers to a situation, wherein the amount of binding of a binding molecule to the first epitope in the target biomolecule comprising the second epitope is at least 50%, particularly at least 75%, and more particularly at least 90% of the amount of binding achieved with a reference construct, where the second epitope is not present.

In certain embodiments, the antibody molecule is able to aggregate a monomeric biomolecule as measured by the following steps: (a) capturing a first, second, and third antibody molecule at the same concentration on the surface of an analytical surface plasmon resonance (“SPR”) instrument, particularly a Biacore™ instrument, wherein said first antibody molecule comprises both said paratopes, wherein said second antibody molecule only comprises said first paratope, and wherein said third antibody only comprises said second paratope, (b) allowing a sample of the monomeric target biomolecule to flow over the captured antibody molecules, and (c) determining the kinetic interaction between the antibody molecules and the monomeric target molecule, wherein the interaction of the first antibody molecule shows a kinetic interaction with the sample of monomeric target biomolecule more typical of a bivalent interaction than the kinetic interaction of said second antibody molecule or the kinetic interaction of said third antibody molecule.

In certain embodiments, the antibody molecule is able to aggregate a monomeric biomolecule as measured by the following steps: (a) immobilizing a first unlabeled version of said antibody molecule in a sandwich ELISA, (b) contacting said immobilized antibody molecule with said soluble monomeric target molecule, (c) permitting the formation of the immobilized antibody molecule and the soluble biomolecule via first paratope/first epitope interaction, and (d) contacting the complexes formed in step (b) with a second version of said antibody molecule, which is labeled or tagged, wherein binding of said second antibody molecule via a second paratope to the second epitope on the immobilized target biomolecule can be detected by identifying the presence of the label or tag of the second version of the claimed antibody molecule.

In certain embodiments, the antibody molecule is able to aggregate a monomeric biomolecule as measured by the following steps: (a) contacting the antibody molecule and the monomeric biomolecule in solution at concentrations, which are at least 5-fold above the estimated or measured K_{D} of the interaction of lowest affinity between the antibody molecule and the epitopes on the target biomolecule; and (b) determining the average molecular weight of the resulting antibody-biomolecule complexes, wherein aggregation is shown by a higher molecular weight of said complexes when compared to the calculated molecular weight of one antibody molecule plus two target molecules, as measured by dynamic light scattering, size exclusion chromatography, analytical ultracentrifugation or another analytical technique.

In other embodiments, the antibody molecule is able to aggregate a monomeric biomolecule as measured by the following steps: (a) contacting said antibody molecule and the monomeric biomolecule in solution at concentrations, which are at least 5-fold above the estimated or measured K_{D} of the interaction of lowest affinity between the antibody molecule and the epitopes on the target biomolecule; (b) and separately contacting a second antibody molecule, having only one of the two paratopes, but having a calculated molecular weight at least as high as said antibody molecule comprising both paratopes, with the monomeric biomolecule in solution at said concentrations, and (c) determining the average molecular weights of the resulting antibody-biomolecule complexes, wherein aggregation is shown when the measured average molecular weight of the resulting antibody-target biomolecule complexes for the antibody comprising both paratopes exceeds the measured average molecular weight of the resulting antibody-target biomolecule complexes for the antibody comprising only one paratope by more than the calculated molecular weight of the target molecule, as measured by dynamic light scattering, size exclusion chromatography, analytical ultracentrifugation or another analytical technique.

In yet other embodiments, the antibody molecule is able to form multimeric immune complexes with said monomeric target biomolecule, which are able to multivalently bind to multivalent mammalian complement proteins, particularly C1q, as measured by the following steps: (a) injecting a mammal with labeled monomeric target biomolecule and with said antibody molecule comprising two paratopes, in such a way that the expected resulting serum concentrations of the antibody and of the target molecule are both simultaneously at least 5-fold above the K_{D} values of the interactions between said antibody and said two epitopes, (b) detecting the label in the liver of the mammal, wherein an at least 2-fold higher signal is obtained when compared to the signal from a control antibody molecule comprising only one of the two said paratopes injected in the same way.

In particular embodiments, the concentrations are 100 μM.

In another aspect, the present invention relates to an antibody molecule comprising at least two independent paratopes, wherein the first paratope is able to specifically bind a first epitope present on monomeric soluble target molecule and the second paratope is able to specifically bind a second epitope present on a multimeric soluble target molecule.

In one embodiment, the antibody molecule is able to bind said monomeric target biomolecule and said multimeric target molecule simultaneously, particularly as demonstrated by a biochemical analysis method, particularly by SPR or sandwich ELISA analysis.

In certain embodiments, the monomeric soluble target biomolecule and the multimeric soluble target molecule are both implicated in the same disease.

In certain embodiments, the monomeric soluble target biomolecule and the multimeric soluble target molecule are both human cytokines.

In certain other embodiments, the monomeric soluble target biomolecule is human GM-CSF and the multimeric soluble target molecule is human TNF-alpha.

In particular embodiments, the monomeric soluble target biomolecule is human IL-6 and the multimeric soluble target molecule is human TNF-alpha.

In particular embodiments, the monomeric soluble target biomolecule is human IL-6 and the multimeric soluble target molecule is human VEGF165.

In particular embodiments, the antibody molecule is a bi-specific antibody.

In particular embodiments, the binding molecule having at least two different binding sites further comprises an Fc region.

In particular such embodiments, the at least one binding site of the binding molecule is comprised in an antigen-binding region of an antibody. In particular embodiments, said at least two binding site of the binding molecule are both comprised in an antigen-binding region of an antibody.

In other particular embodiments, the at least one binding site of the binding molecule is comprised in a binding site different from an antigen-binding region of an antibody. In particular embodiments, said at least two binding site of the binding molecule are both comprised in a binding site different from an antigen-binding region of an antibody.

In particular such embodiments, the Fc region of said binding molecule is a human IgG1 Fc region.

In particular embodiments, at least one of the paratopes of said binding molecule binds to the corresponding epitope on said soluble monomeric biomolecule in a way, which inhibits binding of said epitope to a native binding partner required for signalling.

In particular embodiments, both paratopes of said binding molecule bind to their respective epitopes on said soluble monomeric biomolecule in a way, which inhibit binding of said epitopes to their native binding partners required for signalling. In the context of the present invention, such epitopes are called “inhibitory epitopes”.

Many biomolecules require binding to cognate ligands and/or cell-bound receptors via at least two interactions for signalling. Binding to one of the biomolecule sites required for signalling is able to inhibit signalling. However, binding events are equilibrium reactions, so that at least a certain fraction of the bound biomolecule is always available for signalling, depending on the equilibrium constant. In essence, the complexes formed from biomolecule and inhibitory molecule that are present in the blood are a constant source of at least low amounts of biomolecule available for signalling. By using bi-specific constructs as contemplated by the present invention, wherein both specificities are directed at inhibitory epitopes, the presence of free biomolecules is largely prevented, since the simultaneous dissociation of both inhibitory constructs would be required for generating a free biomolecule.

In a particular embodiment of the present invention, the soluble monomeric biomolecule is IL6, and the binding molecule is a bi-specific antibody molecule, or a functional fragment of an antibody molecule, with two paratopes specific for two different inhibitory epitopes of IL6, wherein said antibody molecule or functional fragment thereof further comprises at least an Fc region.

In a particular embodiment, the bi-specific antibody molecule, or functional fragment thereof comprises variable domain sequences selected from the sequences shown in Table 1.

In another aspect, the present invention relates to a pharmaceutical composition comprising the antibody molecule of the present invention, and optionally a pharmaceutically acceptable carrier and/or excipient.

In yet another aspect, the present invention relates to a binding molecule comprising at least two different binding sites, wherein at least one binding site is specific for an epitope present on a soluble monomeric target biomolecule, for use in removing said target biomolecule from a bodily fluid, wherein said removal occurs by the formation of multimeric complexes comprising said binding molecule and said target biomolecule.

In one embodiment, the binding molecule is an antibody molecule of the present invention.

In another aspect, the present invention relates to a pharmaceutical composition comprising the binding molecule of the present invention, and optionally a pharmaceutically acceptable carrier and/or excipient.

The phrase “pharmaceutically acceptable”, as used in connection with pharmaceutical compositions of the invention, refers to molecular entities and other ingredients of such compositions that are physiologically tolerable and do not typically produce untoward reactions when administered to a mammal (e.g., a human). The term “pharmaceutically acceptable” may also mean approved by a regulatory agency of the Federal or a state government or listed in the U.S. Pharmacopeia or other generally recognized pharmacopeia for use in mammals, and more particularly in humans. The compositions may be formulated e.g. for once-a-day administration, twice-a-day administration, or three times a day administration.

The term “carrier” applied to pharmaceutical compositions of the invention refers to a diluent, excipient, or vehicle with which an active compound (e.g., the bispecific antibody molecule) is administered. Such pharmaceutical carriers may be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Suitable pharmaceutical carriers are described in “Remington's Pharmaceutical Sciences” by A.R. Gennaro, 20th Edition.

The active ingredient (e.g., the binding molecule) or the composition of the present invention may be used for the treatment of at least one of the mentioned disorders, wherein the treatment is adapted to or appropriately prepared for a specific administration as disclosed herein (e.g., to once-a-day, twice-a-day, or three times a day administration). For this purpose the package leaflet and/or the patient information contains corresponding information.

The active ingredient (e.g., the bispecific antibody or fragment thereof) or the composition of the present invention may be used for the manufacture of a medicament for the treatment of at least one of the mentioned disorders, wherein the medicament is adapted to or appropriately prepared for a specific administration as disclosed herein (e.g., to once-a-day, twice-a-day, or three times a day administration). For this purpose the package leaflet and/or the patient information contains corresponding information.

### EXAMPLES

The following examples illustrate the invention without limiting its scope.

### Example 1: Generation and Use of Bispecific Antibodies for the Removal of Soluble Monomeric Biomolecules

A highly preferred embodiment of the invention is to build a bi-specific antibody comprising an Fc region against the two epitopes on the monomeric target or against one epitope on the monomeric target and one epitope on a multimeric soluble target that may serve as an vehicle to aggregate the monomeric target. In less preferred embodiments, other multi-specific antibodies or other binders based on alternative scaffolds such as anticalins and DARPINs and preferably fused to an Fc region are built. The highly preferred bi-specific antibodies according to the invention may be discovered as follows.

Animals may be immunized with the monomeric target of interest, or libraries of antibodies may be selected against the monomeric target of interest. In the embodiment where a multimeric target is chosen as vehicle to achieve aggregation of the monomeric target of interest, separate animals are also immunized with the multimeric target or antibody libraries are also separately selected against the multimeric target. From immunized animals, hybridoma cell lines secreting monoclonal antibodies are generated using standard methods, while with library approaches, selected clones are expressed as soluble antibodies, soluble antibody fragments such as single chain Fvs, Fabs or domain antibodies, produced as antibody-on-phage particles or generated in another manner suitable for specificity screening. For any of the routes chosen to generate binders against the monomeric target of interest and optionally against the multimeric target chosen as aggregation vehicle, produced antibodies are screened for specificity. This is done using standard immunological assays such as enzyme-linked immunosorbant assay (ELISA) or biochemical assays such as surface plasmon resonance (SPR). Once specific clones have been identified against the monomeric target of interest or the multimeric target used as aggregation vehicle, epitopes are characterised in the most preferred embodiments as follows:

In one embodiment it is decided to produce a bi-specific antibody against two epitopes on the monomeric target. In a highly preferred embodiment, the two epitopes on the monomeric target do not overlap. In the most preferred embodiment, both epitopes are inhibitory epitopes, characterised by the fact that when the monomeric target is bound by an antibody on this epitope it is no longer able to perform its natural function such as interaction with a receptor component, or signaling complex formation. Antibodies able to bind two epitopes on the same copy of the monomeric target molecule are identified using immunological methods such as competition ELISA or biochemical methods such as competition studies or additive binding studies on an SPR instrument such as a Biacore™. In this way, monospecific antibody clones directed against different epitopes on the monomeric target are identified. The clones may be grouped into different epitope bins, which are sets of binders that compete strongly with one another for binding to the monomeric target of interest.

Following epitope binning, in a preferred embodiment, two antibody clones from different bins are chosen which show little competition, and in the most preferred embodiment, two antibodies are chosen that show no competition for binding to the monomeric target. These two clones are then converted into a bi-specific antibody format as described herein, preferably one comprising an Fc region. Preferably, the ability of the bispecific antibody molecule to aggregate the monomeric target of interest is then tested. Suitable tests include Dynamic light scattering (DLS), Size-exclusion high-performance liquid chromatography (SEC-HPLC), multi-angle laser light scattering (MALLS) and analytical ultracentrifugation. Sufficiently high concentrations of the bispecific antibody and the monomeric target need to be used to allow aggregation to be measured. Preferably, aggregation measurements are performed with the antibody and target being present at concentrations above the KD of the interaction between the antibody and the monomeric target. For antibodies aimed at therapeutic applications, immune complex formation between the antibody and the monomeric target of interest may be assessed by verifying that the antibody clears a labeled version of the monomeric target from a bodily fluid. A preferred example of such a test is where an animal is injected with both the labeled target of interest and the antibody, and where it is verified that with the bispecific antibody according to the present invention the label appears more and/or faster in the liver of the animal than with a control antibody.

For antibodies aimed at therapeutic applications, the antibodies may be optimized before or after the step of converting monospecific antibodies into bi-specific antibodies. Optimizations steps may comprise but are not limited to humanization and affinity maturation.

In a second embodiment it is decided to produce a bi-specific antibody against one epitope on the monomeric target of interest and against one epitope on a multimeric target that may be used as a vehicle to aggregate the monomeric target of interest. For bi-specific antibodies aimed at therapeutic applications, in a preferred embodiment the monomeric target and the multimeric target are both implicated in the same disease against which the treatment is directed. In a highly preferred embodiment, the epitopes on the monomeric target and the multimeric target are both inhibitory epitopes, characterised by the fact that when the monomeric target is bound by an antibody on this epitope it is no longer able to perform its natural function such as interaction with a receptor component, or signaling complex formation.

In the second embodiment in a next step, the monospecific antibodies directed against the epitope on the monomeric target and against the epitope on the multimeric target are then converted into a bi-specific antibody format as described below, preferably one comprising an Fc region. The final format should allow the bispecific antibody molecule to engage the two selected epitopes simultaneously, allowing the antibody molecule to cross-link the monomeric target of interest and the multimeric target chosen as aggregation vehicle. Such simultaneous engagement can be verified using immunological methods such as competition ELISA or biochemical methods such as competition studies or additive binding studies on an SPR instrument such as a Biacore™.

Preferably, the ability of the bispecific antibody molecule to cross-link the monomeric target of interest and the multimeric target is then tested. Suitable tests include Dynamic light scattering (DLS), size-exclusion high-performance liquid chromatography (SEC-HPLC), multi-angle laser light scattering (MALLS) and analytical ultracentrifugation. Sufficiently high concentrations of the bispecific antibody and the two targets need to be used to allow aggregation to be measured. Preferably, aggregation measurements are performed with the antibody and target being present at concentrations above both the KDs of the interactions between the antibody and the monomeric, and between the antibody and the multimeric target. For antibodies aimed at therapeutic applications, immune complex formation between the antibody, the monomeric target of interest and the multimeric target used as an aggregation vehicle may be assessed by verifying that the antibody clears a labeled version of the monomeric target from a bodily fluid. A preferred example of such a test is where an animal is injected with the labeled monomeric target of interest, the multimeric target and the antibody, and where it is verified that with the bispecific antibody according to the present invention the label appears more and/or faster in the liver of the animal than with a control antibody.

For antibodies aimed at therapeutic applications, the antibodies may be optimized before or after the step of converting monospecific antibodies into bi-specific antibodies. Optimizations steps may comprise but are not limited to humanization and affinity maturation.

### Example 2: Cloning and Production of Fabs in E. coli

Fab fragments of two monospecific human IgG1 antibodies against IL6 were produced, Mab4 (with variable domains as listed in WO2007076927) and Mab5 (with variable domains as listed in WO2011066371). Synthetic cDNAs encoding Fab fragments of Mab4 and Mab5 were generated and cloned into an E. coli expression vector in the context of cDNAs encoding heavy and light chain secretory signal peptides and a polyhistidine tag, which was fused to the heavy chain CH1 domain. Expression constructs were transformed into TG1 cells and production carried out as follows: Clones bearing Fab expression constructs were grown in LB and TB solid and liquid media, purchased from Carl Roth, which were supplemented with Carbenicillin and glucose, purchased from VWR. Antibody expression in liquid cultures was performed overnight in Erlenmeyer flasks in a shaking incubator and was induced by the addition of isopropyl-β-D-thiogalactopyranoside (IPTG), purchased from Carl Roth, to the growth medium. Culture supernatants containing secreted Fab fragments were clarified by centrifugation of the expression cultures. Expressed Fab fragments were then purified from the culture supernatant in a standard immobilized-metal affinity chromatography (IMAC) procedure, using NiNTA resin purchased from Qiagen. Fab fragments were eluted from the NiNTA resin using a buffer composed of 75 mM EDTA and 75 mM TrisHCl, pH6.8. Purified Fab fragments were buffer-exchanged into HBS-EP+buffer using illustra NAP-5 desalting columns, both purchased from GE Healthcare, according to manufacturer's instructions.

### Example 3: Co-Binding of Fab5 and Fab4

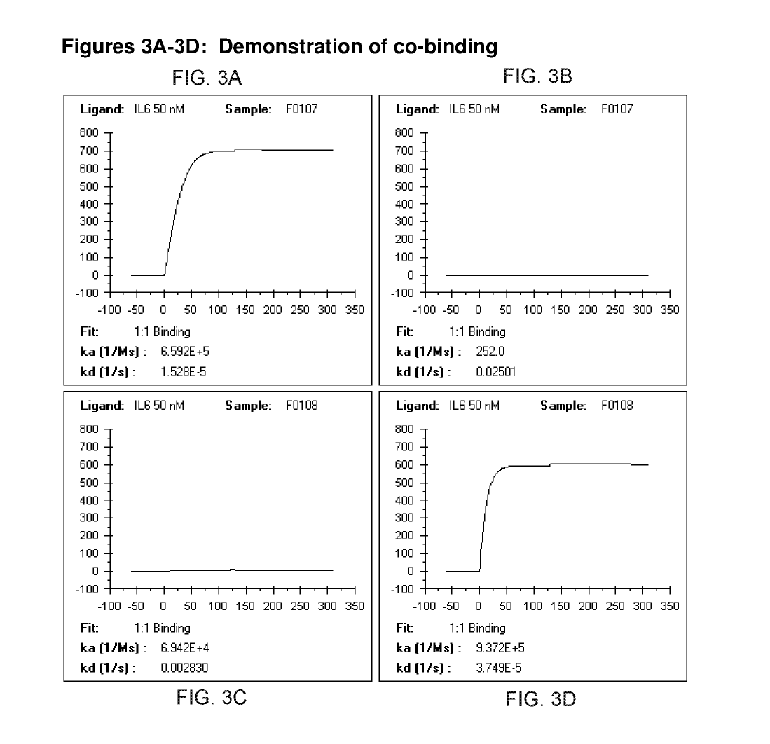

In order to demonstrate the suitability of the anti-IL6 antibodies Mab4 and Mab5 as modules for the construction of bispecific antibodies, co-binding of Fab fragments of the two antibodies to IL6 was examined by Biacore. For this, Fab4 and Fab5 were immobilized onto a Biacore CM5 chip at 400 RU and 2140 RU, respectively, using amine coupling. Onto these immobilized Fab fragments IL6 was captured resulting in 340 RU and 120 RU for the Fab5 and Fab4, respectively. As can be seen in FIG. 3, flowing 100 nM Fab fragment of the two antibodies over the surface demonstrate that these two antibodies bind non-overlapping epitopes on IL6.

### Example 4: Production of Monospecific and Bispecific IgG Antibodies Against Human IL-6

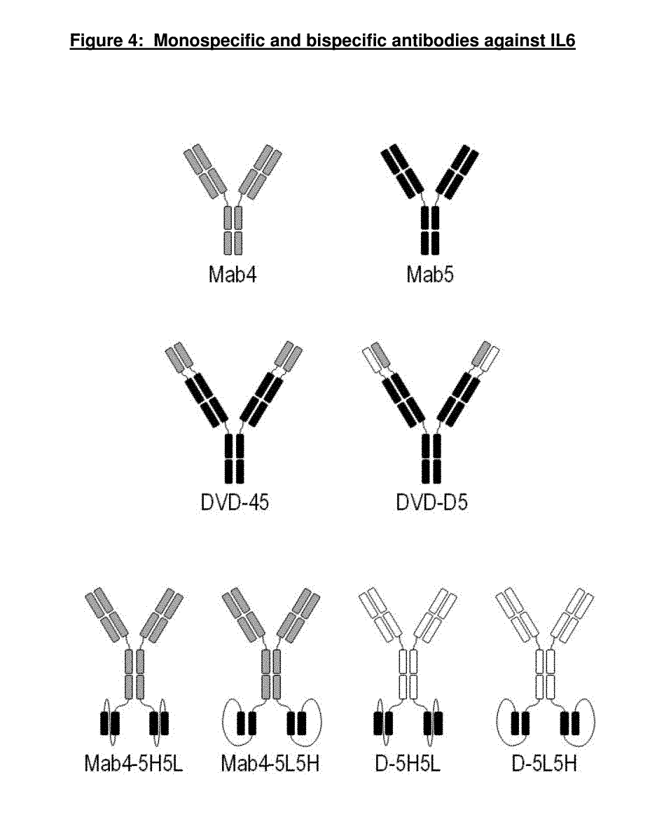

Antibodies were produced against human IL-6 as an exemplary monomeric target protein. The exemplary antibody sequences used are listed in Table 1 and the constructs are illustrated in FIG. 4. Two monospecific human IgG1 antibodies against IL6 were produced, Mab4 (with variable domains as listed in WO2007076927) and Mab5 (with variable domains as listed in WO2011066371), because it had been demonstrated (see example 2) that these two antibodies bind non-overlapping epitopes on human IL6. Bispecific, tetravalent human IgG1 antibodies comprising the same variable domain sequences were constructed in several formats. First, the antibody DVD-45 is a dual variable domain IgG, in which the variable domains of Mab4 are appended to the N-termini of the variable domains of Mab5, using a 9-amino-acid linker. Second, the antibody Mab4-5H5L is an IgG-single chain Fv fusion, in which the VH domain of Mab5 is fused to the C-terminus of the CH3 domain of Mab4 using a 7-amino-acid linker, and the VL domain of Mab5 is fused to the VH domain of Mab5 using a 15-amino-acid linker. Third, the antibody Mab4-5L5H is an IgG-single chain Fv fusion, in which the VL domain of Mab5 is fused to the C-terminus of the CH3 domain of Mab4 using a 7-amino-acid linker, and the VH domain of Mab5 is fused to the VL domain of Mab5 using a 16-amino-acid linker. Furthermore, monospecific control constructs with identical domain arrangements to the bispecific antibodies were constructed. The first monospecific control used, antibody DVD-D5, is a dual variable domain IgG similar to DVD-45 but with the light chain variable domain of Mab4 replaced with a germline-like dummy light chain variable domain, therefore comprising only one pair of anti-IL6 binding sites, namely the Mab5 variable regions, but within the same domain arrangement as in antibody DVD-45. The second monospecific control used, antibody D-5H5L is an IgG-single chain Fv fusion, in which the VH domain of Mab5 is fused to the C-terminus of the CH3 domain of a Dummy antibody with germline-like variable domains, using a 7-amino-acid linker, and the VL domain of Mab5 is fused to the VH domain of Mab5 using a 15-amino-acid linker. The third monospecific control used, antibody D-5L5H is an IgG-single chain Fv fusion, in which the VL domain of Mab5 is fused to the C-terminus of the CH3 domain of a Dummy antibody with germline-like variable domains, using a 7-amino-acid linker, and the VH domain of Mab5 is fused to the VL domain of Mab5 using a 16-amino-acid linker.

Genes encoding heavy or light chains of these monospecific and bispecific antibodies were constructed by gene synthesis and cloned into the mammalian expression vector pTT5, modified by the addition of sequences encoding mammalian secretory signal peptides. To produce antibodies, expression constructs encoding heavy and light chains were transiently co-transfected into CHO cells and cells were maintained for 5 days in serum-free suspension cultures. Cell culture supernatants were clarified by centrifugation and antibodies affinity-purified using protein A resin (ProSep vA Ultra, Millipore catalogue number 115115827). Antibodies were eluted using a buffer comprising 10 mM citric acid, 70 mM NaCl and 4% v/v glycerol, and neutralized by addition of a 8% volume Tris HCl pH8.0. For cell assays, antibody stocks were buffer-exchanged into PBS pH7.4 (catalogue number 10010) supplemented with 4% glycerol, using illustra NAP-5 columns (GE Healthcare catalogue number 17-0853-02). For complement assays, affinity-purified antibodies were further purified by preparative SEC-HPLC using a running buffer of 1×PBS pH7.4 (prepared from 10× stock, Gibco catalogue number 70011), supplemented with 3% ethanol, and used in complement assays within 24 hours.

TABLE 1
Sequences of IgG antibodies against IL6

| | Heavy | |
|---|---|---|
| | Chain/ | |
| | Light | |
| Antibody | Chain | Amino acid sequence |
| Mab4 | HC | EVKFEESGGGLVQPGGSMKLSCVASGFSFSNYWMNWV |
| | | RQSPEKGLEWVAEIRLTSNKQAIYYAESVKGRFTISRDD |
| | | SKSSVYLQMNNLRAEDTGIYYCASLFYDGYLHWGQGTL |
| | | VTVSSASTKGPSVFPLAPSSKSTSGGTAALGCLVKDYFPE |
| | | PVTVSWNSGALTSGVHTFPAVLQSSGLYSLSSVVTVPSS |
| | | SLGTQTYICNVNHKPSNTKVDKKVEPKSCDKTHTCPPCP |
| | | APELLGGPSVFLFPPKPKDTLMISRTPEVTCVVVDVSHED |
| | | PEVKFNWYVDGVEVHNAKTKPREEQYNSTYRVVSVLT |
| | | VLHQDWLNGKEYKCKVSNKALPAPIEKTISKAKGQPRE |
| | | PQVYTLPPSRDELTKNQVSLTCLVKGFYPSDIAVEWESN |
| | | GQPENNYKTTPPVLDSDGSFFLYSKLTVDKSRWQQGNV |
| | | FSCSVMHEALHNHYTQKSLSLSPGK |
| Mab4 | LC | DIVLTQSPASLAVSLGQRATISCRASESVGNFGISFMNWF |
| | | QQKPGQPPKLLIYTASNQGSGVPARFSGSGSGTDFSLNIH |
| | | PMEEDDSAMYFCQQSKEIPWTFGGGTKVEIKRTVAAPS |
| | | VFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSFNRGEC |
| Mab5 | HC | EVQLVESGGGLVQPGGSLRLSCAASGFSLSNYYVTWVR |
| | | QAPGKGLEWVGIIYGSDETAYATSAIGRFTISRDNSKNTL |
| | | YLQMNSLRAEDTAVYYCARDDSSDWDAKFNLWGQGT |
| | | LVTVSSASTKGPSVFPLAPSSKSTSGGTAALGCLVKDYFP |
| | | EPVTVSWNSGALTSGVHTFPAVLQSSGLYSLSSVVTVPS |
| | | SSLGTQTYICNVNHKPSNTKVDKKVEPKSCDKTHTCPPC |
| | | PAPELLGGPSVFLFPPKPKDTLMISRTPEVTCVVVDVSHE |
| | | DPEVKFNWYVDGVEVHNAKTKPREEQYNSTYRVVSVL |
| | | TVLHQDWLNGKEYKCKVSNKALPAPIEKTISKAKGQPR |
| | | EPQVYTLPPSRDELTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGK |
| Mab5 | LC | AIQMTQSPSSLSASVGDRVTITCQASQSINNELSWYQQK |
| | | PGKAPKLLIYRASTLASGVPSRFSGSGSGTDFTLTISSLQP |
| | | DDFATYYCQQGYSLRNIDNAFGGGTKVEIKRTVAAPSV |
| | | FIFPPSDEQLKSGTASVVCLLNNFYPREAKVQWKVDNAL |
| | | QSGNSQESVTEQDSKDSTYSLSSTLTLSKADYEKHKVYA |
| | | CEVTHQGLSSPVTKSFNRGEC |
| DVD-45 | HC | EVKFEESGGGLVQPGGSMKLSCVASGFSFSNYWMNWV |
| | | RQSPEKGLEWVAEIRLTSNKQAIYYAESVKGRFTISRDD |
| | | SKSSVYLQMNNLRAEDTGIYYCASLFYDGYLHWGQGTL |
| | | VTVSSPAPNLLGGPEVQLVESGGGLVQPGGSLRLSCAAS |
| | | GFSLSNYYVTWVRQAPGKGLEWVGIIYGSDETAYATSAI |
| | | GRFTISRDNSKNTLYLQMNSLRAEDTAVYYCARDDSSD |
| | | WDAKFNLWGQGTLVTVSSASTKGPSVFPLAPSSKSTSG |
| | | GTAALGCLVKDYFPEPVTVSWNSGALTSGVHTFPAVLQ |
| | | SSGLYSLSSVVTVPSSSLGTQTYICNVNHKPSNTKVDKK |
| | | VEPKSCDKTHTCPPCPAPELLGGPSVFLFPPKPKDTLMIS |
| | | RTPEVTCVVVDVSHEDPEVKFNWYVDGVEVHNAKTKP |
| | | REEQYNSTYRVVSVLTVLHQDWLNGKEYKCKVSNKAL |
| | | PAPIEKTISKAKGQPREPQVYTLPPSRDELTKNQVSLTCL |
| | | VKGFYPSDIAVEWESNGQPENNYKTTPPVLDSDGSFFLY |
| | | SKLTVDKSRWQQGNVFSCSVMHEALHNHYTQKSLSLSP |
| | | GK |
| DVD-45 | LC | DIVLTQSPASLAVSLGQRATISCRASESVGNFGISFMNWF |
| | | QQKPGQPPKLLIYTASNQGSGVPARFSGSGSGTDFSLNIH |
| | | PMEEDDSAMYFCQQSKEIPWTFGGGTKLEIKSPAPNLLG |
| | | GPAIQMTQSPSSLSASVGDRVTITCQASQSINNELSWYQ |
| | | QKPGKAPKLLIYRASTLASGVPSRFSGSGSGTDFTLTISSL |
| | | QPDDFATYYCQQGYSLRNIDNAFGGGTKVEIKRTVAAP |
| | | SVFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSFNRGEC |
| DVD-D5 | HC | EVKFEESGGGLVQPGGSMKLSCVASGFSFSNYWMNWV |
| | | RQSPEKGLEWVAEIRLTSNKQAIYYAESVKGRFTISRDD |
| | | SKSSVYLQMNNLRAEDTGIYYCASLFYDGYLHWGQGTL |
| | | VTVSSPAPNLLGGPEVQLVESGGGLVQPGGSLRLSCAAS |
| | | GFSLSNYYVTWVRQAPGKGLEWVGIIYGSDETAYATSAI |
| | | GRFTISRDNSKNTLYLQMNSLRAEDTAVYYCARDDSSD |
| | | WDAKFNLWGQGTLVTVSSASTKGPSVFPLAPSSKSTSG |
| | | GTAALGCLVKDYFPEPVTVSWNSGALTSGVHTFPAVLQ |
| | | SSGLYSLSSVVTVPSSSLGTQTYICNVNHKPSNTKVDKK |
| | | VEPKSCDKTHTCPPCPAPELLGGPSVFLFPPKPKDTLMIS |
| | | RTPEVTCVVVDVSHEDPEVKFNWYVDGVEVHNAKTKP |
| | | REEQYNSTYRVVSVLTVLHQDWLNGKEYKCKVSNKAL |
| | | PAPIEKTISKAKGQPREPQVYTLPPSRDELTKNQVSLTCL |
| | | VKGFYPSDIAVEWESNGQPENNYKTTPPVLDSDGSFFLY |
| | | SKLTVDKSRWQQGNVFSCSVMHEALHNHYTQKSLSLSP |
| | | GK |
| DVD-D5 | LC | DTQMTQSPSSLSASVGDRVTITCRASQSISSYLAWYQQK |
| | | PGKAPKLLIYAASSLYSGVPSRFSGSGSGTDFTLTISSLQP |
| | | EDFATYYCQQYSSLPYTFGQGTKLEIKSPAPNLLGGPAIQ |
| | | MTQSPSSLSASVGDRVTITCQASQSINNELSWYQQKPGK |
| | | APKLLIYRASTLASGVPSRFSGSGSGTDFTLTISSLQPDDF |
| | | ATYYCQQGYSLRNIDNAFGGGTKVEIKRTVAAPSVFIFP |
| | | PSDEQLKSGTASVVCLLNNFYPREAKVQWKVDNALQSG |
| | | NSQESVTEQDSKDSTYSLSSTLTLSKADYEKHKVYACEV |
| | | THQGLSSPVTKSFNRGEC |
| Mab4-5H5L | HC | EVKFEESGGGLVQPGGSMKLSCVASGFSFSNYWMNWV |
| | | RQSPEKGLEWVAEIRLTSNKQAIYYAESVKGRFTISRDD |
| | | SKSSVYLQMNNLRAEDTGIYYCASLFYDGYLHWGQGTL |
| | | VTVSSASTKGPSVFPLAPSSKSTSGGTAALGCLVKDYFPE |
| | | PVTVSWNSGALTSGVHTFPAVLQSSGLYSLSSVVTVPSS |
| | | SLGTQTYICNVNHKPSNTKVDKKVEPKSCDKTHTCPPCP |
| | | APELLGGPSVFLFPPKPKDTLMISRTPEVTCVVVDVSHED |
| | | PEVKFNWYVDGVEVHNAKTKPREEQYNSTYRVVSVLT |
| | | VLHQDWLNGKEYKCKVSNKALPAPIEKTISKAKGQPRE |
| | | PQVYTLPPSRDELTKNQVSLTCLVKGFYPSDIAVEWESN |
| | | GQPENNYKTTPPVLDSDGSFFLYSKLTVDKSRWQQGNV |
| | | FSCSVMHEALHNHYTQKSLSLSPGSGSASGGSEVQLVES |
| | | GGGLVQPGGSLRLSCAASGFSLSNYYVTWVRQAPGKGL |
| | | EWVGIIYGSDETAYATSAIGRFTISRDNSKNTLYLQMNSL |
| | | RAEDTAVYYCARDDSSDWDAKFNLWGQGTLVTVSSGG |
| | | GGSGGGGSGGGGSAIQMTQSPSSLSASVGDRVTITCQAS |
| | | QSINNELSWYQQKPGKAPKLLIYRASTLASGVPSRFSGS |
| | | GSGTDFTLTISSLQPDDFATYYCQQGYSLRNIDNAFGGG |
| | | TKVEIK |
| Mab4-5H5L | LC | DIVLTQSPASLAVSLGQRATISCRASESVGNFGISFMNWF |
| | | QQKPGQPPKLLIYTASNQGSGVPARFSGSGSGTDFSLNIH |
| | | PMEEDDSAMYFCQQSKEIPWTFGGGTKVEIKRTVAAPS |
| | | VFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSFNRGEC |
| Mab4-5L5H | HC | EVKFEESGGGLVQPGGSMKLSCVASGFSFSNYWMNWV |
| | | RQSPEKGLEWVAEIRLTSNKQAIYYAESVKGRFTISRDD |
| | | SKSSVYLQMNNLRAEDTGIYYCASLFYDGYLHWGQGTL |
| | | VTVSSASTKGPSVFPLAPSSKSTSGGTAALGCLVKDYFPE |
| | | PVTVSWNSGALTSGVHTFPAVLQSSGLYSLSSVVTVPSS |
| | | SLGTQTYICNVNHKPSNTKVDKKVEPKSCDKTHTCPPCP |
| | | APELLGGPSVFLFPPKPKDTLMISRTPEVTCVVVDVSHED |
| | | PEVKFNWYVDGVEVHNAKTKPREEQYNSTYRVVSVLT |
| | | VLHQDWLNGKEYKCKVSNKALPAPIEKTISKAKGQPRE |
| | | PQVYTLPPSRDELTKNQVSLTCLVKGFYPSDIAVEWESN |
| | | GQPENNYKTTPPVLDSDGSFFLYSKLTVDKSRWQQGNV |
| | | FSCSVMHEALHNHYTQKSLSLSPGSGSASGGSAIQMTQS |
| | | PSSLSASVGDRVTITCQASQS1NNELSWYQQKPGKAPKL |
| | | LIYRASTLASGVPSRFSGSGSGTDFTLTISSLQPDDFATYY |
| | | CQQGYSLRNIDNAFGGGTKVEIKSGGGGSGGGGSGGGG |
| | | SEVQLVESGGGLVQPGGSLRLSCAASGFSLSNYYVTWV |
| | | RQAPGKGLEWVGIIYGSDETAYATSAIGRFTISRDNSKNT |
| | | LYLQMNSLRAEDTAVYYCARDDSSDWDAKFNLWGQG |
| | | TLVTVSS |
| Mab4-5L5H | LC | DIVLTQSPASLAVSLGQRATISCRASESVGNFGISFMNWF |
| | | QQKPGQPPKLLIYTASNQGSGVPARFSGSGSGTDFSLNIH |
| | | PMEEDDSAMYFCQQSKEIPWTFGGGTKVEIKRTVAAPS |
| | | VFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSFNRGEC |
| D-5H5L | HC | EVQLVESGGGLVQPGGSLRLSCAASGFTFSSYAMSWIRQ |
| | | APGKGLEWIGQISGSGGSTYYNDNVLGRFTISRDNSKNT |
| | | LYLQMNSLRAEDTAVYYCARDSGYFDIWGQGTLVTVSS |
| | | ASTKGPSVFPLAPSSKSTSGGTAALGCLVKDYFPEPVTVS |
| | | WNSGALTSGVHTFPAVLQSSGLYSLSSVVTVPSSSLGTQ |
| | | TYICNVNHKPSNTKVDKKVEPKSCDKTHTCPPCPAPELL |
| | | GGPSVFLFPPKPKDTLMISRTPEVTCVVVDVSHEDPEVK |
| | | FNWYVDGVEVHNAKTKPREEQYNSTYRVVSVLTVLHQ |
| | | DWLNGKEYKCKVSNKALPAPIEKTISKAKGQPREPQVY |
| | | TLPPSRDELTKNQVSLTCLVKGFYPSDIAVEWESNGQPE |
| | | NNYKTTPPVLDSDGSFFLYSKLTVDKSRWQQGNVFSCS |
| | | VMHEALHNHYTQKSLSLSPGSGSASGGSEVQLVESGGG |
| | | LVQPGGSLRLSCAASGFSLSNYYVTWVRQAPGKGLEWV |
| | | GIIYGSDETAYATSAIGRFTISRDNSKNTLYLQMNSLRAE |
| | | DTAVYYCARDDSSDWDAKFNLWGQGTLVTVSSGGGGS |
| | | GGGGSGGGGSAIQMTQSPSSLSASVGDRVTITCQASQSI |
| | | NNELSWYQQKPGKAPKLLIYRASTLASGVPSRFSGSGSG |
| | | TDFTLTISSLQPDDFATYYCQQGYSLRNIDNAFGGGTKV |
| | | EIK |
| D-5H5L | LC | DTQMTQSPSSLSASVGDRVTITCRASQSISSYLAWYQQK |
| | | PGKAPKLLIYAASSLYSGVPSRFSGSGSGTDFTLTISSLQP |
| | | EDFATYYCQQYSSLPYTFGQGTKVEIKRTVAAPSVFIFPP |
| | | SDEQLKSGTASVVCLLNNFYPREAKVQWKVDNALQSG |
| | | NSQESVTEQDSKDSTYSLSSTLTLSKADYEKHKVYACEV |
| | | THQGLSSPVTKSFNRGEC |
| D-5L5H | HC | EVQLVESGGGLVQPGGSLRLSCAASGFTFSSYAMSWIRQ |
| | | APGKGLEWIGQISGSGGSTYYNDNVLGRFTISRDNSKNT |
| | | LYLQMNSLRAEDTAVYYCARDSGYFDIWGQGTLVTVSS |
| | | ASTKGPSVFPLAPSSKSTSGGTAALGCLVKDYFPEPVTVS |
| | | WNSGALTSGVHTFPAVLQSSGLYSLSSVVTVPSSSLGTQ |
| | | TYICNVNHKPSNTKVDKKVEPKSCDKTHTCPPCPAPELL |
| | | GGPSVFLFPPKPKDTLMISRTPEVTCVVVDVSHEDPEVK |
| | | FNWYVDGVEVHNAKTKPREEQYNSTYRVVSVLTVLHQ |
| | | DWLNGKEYKCKVSNKALPAPIEKTISKAKGQPREPQVY |
| | | TLPPSRDELTKNQVSLTCLVKGFYPSDIAVEWESNGQPE |
| | | NNYKTTPPVLDSDGSFFLYSKLTVDKSRWQQGNVFSCS |
| | | VMHEALHNHYTQKSLSLSPGSGSASGGSAIQMTQSPSSL |
| | | SASVGDRVTITCQASQSINNELSWYQQKPGKAPKWYR |
| | | ASTLASGVPSRFSGSGSGTDFTLTISSLQPDDFATYYCQQ |
| | | GYSLRNIDNAFGGGTKVEIKSGGGGSGGGGSGGGGSEV |
| | | QLVESGGGLVQPGGSLRLSCAASGFSLSNYYVTWVRQA |
| | | PGKGLEWVGIIYGSDETAYATSAIGRFTISRDNSKNTLYL |
| | | QMNSLRAEDTAVYYCARDDSSDWDAKFNLWGQGTLV |
| | | TVSS |
| D-5L5H | LC | DTQMTQSPSSLSASVGDRVTITCRASQSISSYLAWYQQK |
| | | PGKAPKLLIYAASSLYSGVPSRFSGSGSGTDFTLTISSLQP |
| | | EDFATYYCQQYSSLPYTFGQGTKVEIKRTVAAPSVFIFPP |
| | | SDEQLKSGTASVVCLLNNFYPREAKVQWKVDNALQSG |
| | | NSQESVTEQDSKDSTYSLSSTLTLSKADYEKHKVYACEV |
| | | THQGLSSPVTKSFNRGEC |

### Example 5: Immune Complex Formation Demonstrated by Biophysical Methods

For bispecific antibodies according to the invention, the formation of large immune complexes comprising antibody and the monomeric target protein may be demonstrated by biophysical methods. Suitable methods include size-exclusion high-performance liquid chromatography (hereinafter referred to as SEC-HPLC) as demonstrated in the following example. Size Exclusion Chromatography (SEC) is a common technique for the analysis of proteins and protein complexes in their native state. Proteins are separated on a chromatographic column through which they flow with different rates depending on the size, shape and conformation of the protein molecules or complexes. Generally separated proteins and complexes elute according to their size—large complexes elute first, then intermediate complexes and small complexes as well as individual molecules. The elution is typically monitored by ultra-violet (UV) absorbance.

Suitable alternative methods for measuring the formation of immune complexes between monomeric proteins and bispecific antibodies according to the invention include dynamic light scattering (DLS), analytical ultracentrifugation (AUC) and multi-angle laser light scattering (MALLS), as well as any other methods able to resolve small protein complexes of a hydrodynamic size or molecular weight that corresponds to singular immune complexes, comprising one antibody molecule, from large protein complexes of a hydrodynamic size or molecular weight that corresponds to large immune complexes, comprising two or several antibody molecules.

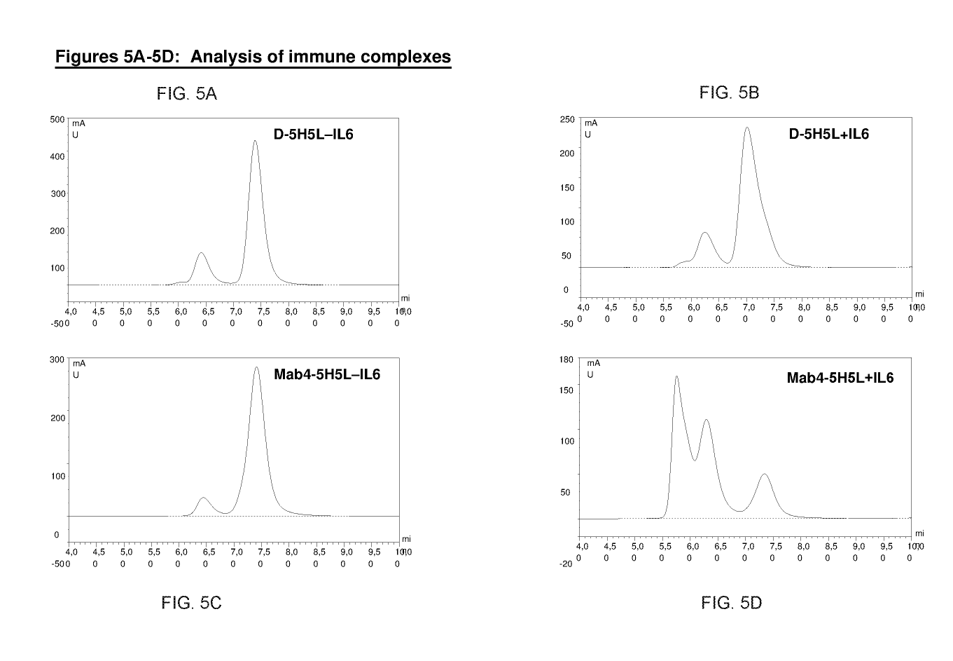

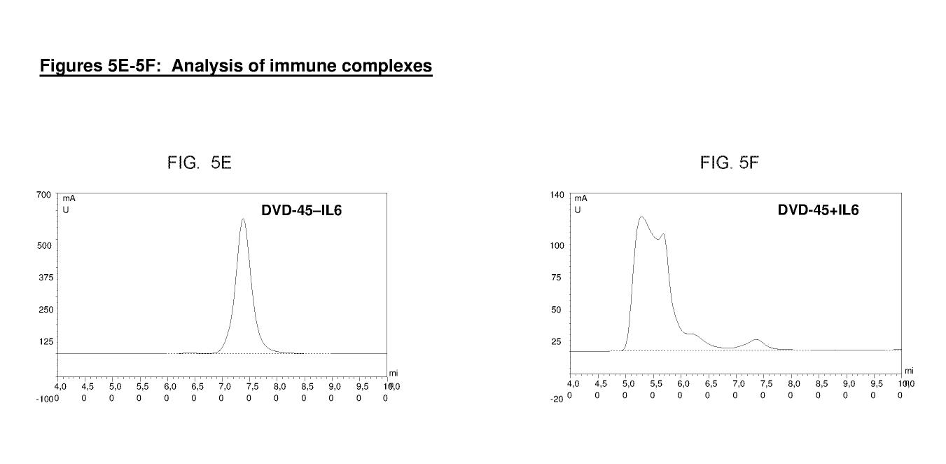

In this example, three purified antibodies against human IL6 were used, each with a molecular weight of approximately 200 kDa and therefore presumed to be of similar hydrodynamic size. The antibodies used were the monospecific bivalent antibody D-5H5L, the bispecific tetravalent antibody DVD-45 and the tetravalent bispecific antibody Mab4-5H5L. The hydrodynamic size of the antibodies was compared either alone or in complex with IL6, using SEC-HPLC. For immune complex formation the purified antibodies were incubated for two hours with purified recombinant human IL6 (Peprotech catalogue number 200-06), by dropwise adding IL6 stock solution to the purified antibody to a final equimolar concentration compared to the calculated concentration of unique antibody binding sites. Following incubation, antibodies with and without IL6 were analysed at room temperature using a TOSOH G3000 SWXL column of 7.8 mm×30 cm, a mobile phase of 10% v/v 10×PBS buffer (Gibco catalogue number 70011), 3% v/v ethanol and 87% water, with a flow rate of 1 ml/min and detection wavelength of 280 nm. The results are illustrated in FIG. 5.

As can be seen from FIGS. 5 A, C and E, the three antibodies without IL6 exhibit a very similar hydrodynamic size and each elute at a retention time of 7.4 minutes, as expected. The antibodies consist mostly of a monomeric fraction as well as a smaller amount of dimer with a retention time of 6.4 minutes, which is a very small fraction of 0.6% in the case of antibody DVD-45, 18.5% in the case of antibody D-5H5L and 7.7% in the case of antibody Mab4-5H5L. This dimeric fraction as well as any aggregates that may be present following antibody production are typically removed during antibody drug manufacturing processes, but if present do not interfere with immune complex analysis, seen in FIGS. 5 B, D and F.

As can be seen by comparing FIG. 5 A with FIG. 5 B, incubation of the monospecific antibody D-5H5L with IL6 results only in a minimal shift of the antibody's retention time, going from 7.4 minutes to 7.0 minutes for the antibodies large monomeric fraction. This corresponds to the shift in molecular weight from approximately 200 kD for the naked antibody to approximately 240 kD for the antibody bound to two IL6 molecules. It is therefore a very clear demonstration of the formation of small, singular immune complexes, comprising only one antibody molecule, between conventional monospecific antibodies and monomeric target proteins. The dimeric fraction of Mab D-5H5L is still 18.5% of total antibody and also exhibits a very slight shift in retention time, moving from 6.4 minutes to 6.2 minutes, as a result of binding up to 4 molecules of IL6 in this analysis.

In contrast, comparison of FIG. 5 C with FIG. 5 D shows a dramatically different result. For the bispecific antibody Mab4-5H5L, a large shift in hydrodynamic size is observed, most of the large monomeric fraction of the antibody has disappeared and only 19.3% remains, being shifted from 7.4 minutes to 7.3 minutes due to binding of IL6. A large 37.5% fraction of Mab4-5H5L now participates in immune complexes that comprise two antibody molecules, indicated by a retention time of 6.3 minutes, and the largest fraction of 43.2% of Mab4-5H5L participates in even larger immune complexes, comprising three or more antibodies and giving a retention time of 5.8 minutes. The example of antibody DVD-45 is even more extreme, as can be seen by comparing FIGS. 5 E and 5 F. For this antibody, a very large part of the monomeric fraction of the antibody has disappeared and only approximately 4.9% remains after immune complex formation. A large 37% fraction of DVD-45 participates in large immune complexes that comprise three or more antibodies, indicated by a peak with a retention time of 5.7 minutes, and the largest fraction of 58% of DVD-45 participates in even larger immune complexes, containing many antibody molecules, with a retention time of approximately 5.3 minutes. The difference observed between antibodies DVD-45 and Mab4-5H5L is a reflection of their different formats, illustrated in FIG. 4, that favor specific stoichiometries in the bispecific binding to the monomeric IL6 protein. It is therefore clear that biophysical methods may not only be used to demonstrate the formation of singular immune complexes between conventional monospecific antibodies and monomeric targets or the formation of large immune complexes between bispecific antibodies and monomeric targets, but may also be used to characterise bispecific antibodies according to the invention and identify bispecific antibody formats or bispecific antibody clones that form particularly large immune complexes.

### Example 6: Removability of Monomeric Targets Demonstrated by Complement C1q Binding

Determination of immune complex binding to complement C1q was performed using ELISA. Human C1q (Calbiochem, 204876) was coated onto maxisorp ELISA plates (NUNC, Denmark) at a concentration of 16 microgram/ml in a buffer containing 10 mM Tris HCl, pH 8 for 1 h and washed twice with 1× phosphate buffered saline supplemented with 0.1% Tween-20 (Merck KGaA, Germany, 8.17072.1000) (PBST). Plates were blocked with 2% skimmed milk powder (Roth, Germany T145.3) in PBST for 30 min and again washed twice with PBST. The prepared plates were then incubated with a preformed complex of antibody and IL6, at an equimolar IL6 and antibody binding site concentration of 11 nM, or with 11 nM antibody incubated without IL6, for 30 min. To determine whether antibodies bound to C1q the plates were then washed twice with PBST, incubated with HRP-Fab′2 donkey anti-human Fc (Jackson ImmunoResearch, 709-036-098, 1:10000) for 30 minutes, and washed six times with PBST and revealed with TMB substrate (KPL, MD, 50-65/76-02).

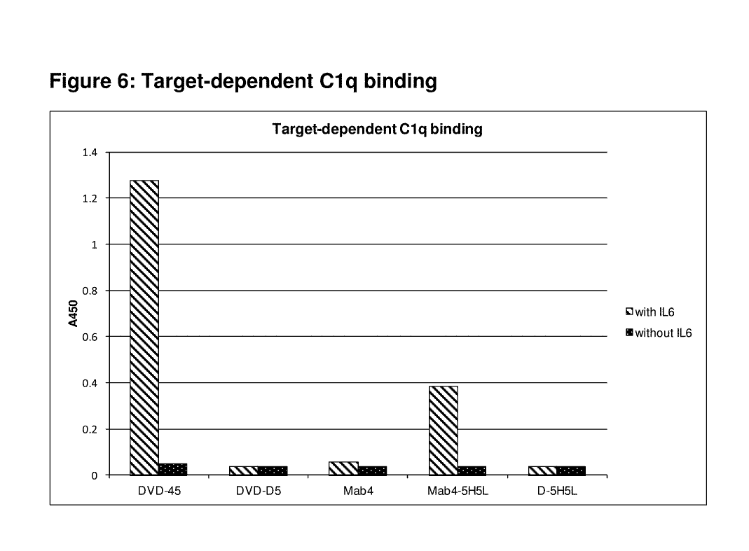

As shown in FIG. 6, exemplary bispecific antibodies against IL6 according to the invention (DVD-45 and Mab4-5L5H) exhibit IL6-dependent formation of large immune complexes that can interact with complement component C1 q, whereas control monospecific antibodies against IL6 (DVD-D5, Mab4, and D-5L5H) do not. Thus, through this target-dependent binding of the bispecific antibody to C1q the target-antibody complex can be cleared from circulation.

### Example 7: Dual Epitope Inhibition in a Cell Based Assay

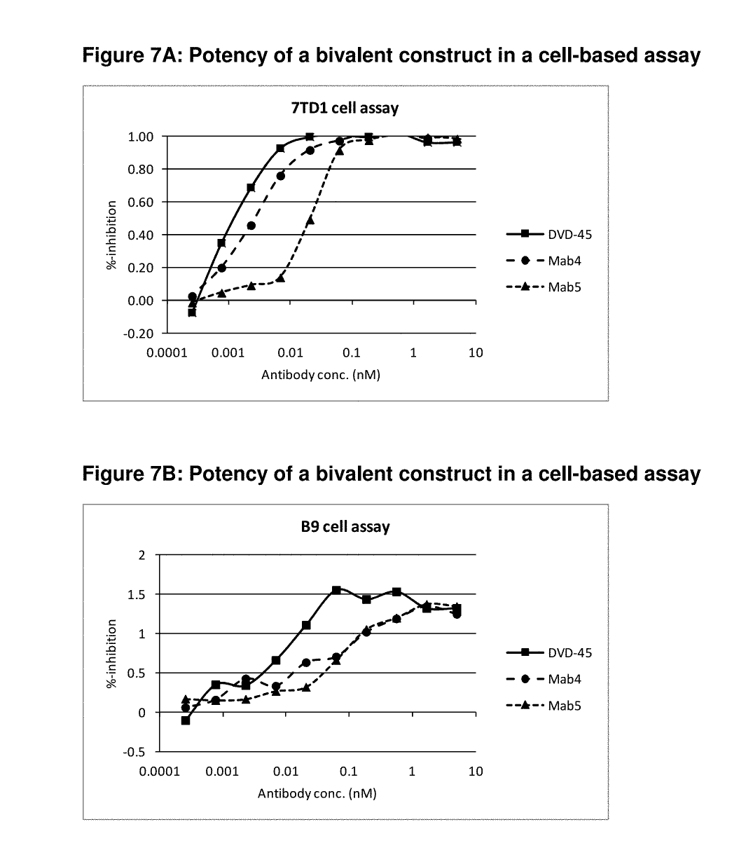

In order to investigate the difference between conventional monospecific antibodies and bispecific antibodies of the invention in their ability to inhibit the biological activity of a monomeric target molecule, bioassays were performed using 7TD1 and B9 cells. Serial dilutions of exemplary conventional antibodies (Mab4 and Mab5) and an exemplary bispecific antibody of the invention (DVD-45) were incubated with a constant concentration of IL6, representing the EC80 on the cell-line used. Each IL6-responsive cell line was then incubated with the mix of antibody and IL6, and the growth-stimulating action of IL6 measured in a proliferation assay. As shown in FIGS. 7a and 7b and in Table 2, the bispecific antibody of the invention, DVD-45, had a significantly lower IC₅₀ and therefore demonstrated greater potency than the conventional monospecific antibodies. In cell assays such as these, the novel ability of bispecific antibodies according to the invention to remove monomeric targets from bodily fluids in vivo in an Fc-dependent manner does not play a role. Rather, the increased potency in these cell assays demonstrates that dual, independent blocking of two inhibitory epitopes on a monomeric target protein is a mechanism by which the novel bispecific antibodies of the invention can be more effective than conventional monospecific antibodies.

TABLE 2

| | IC₅₀ on 7TD1 cells (pM) | IC50 on B9 cells (pM) |
|---|---|---|
| DVD-45 | 1.3 | 2.9 |
| Mab4 | 2.6 | 8.4 |
| Mab5 | 19.9 | 26.1 |

### Example 8: PKPD Modeling to Compare the Effects of a Conventional Antibody with a Bispecific Antibody of the Invention

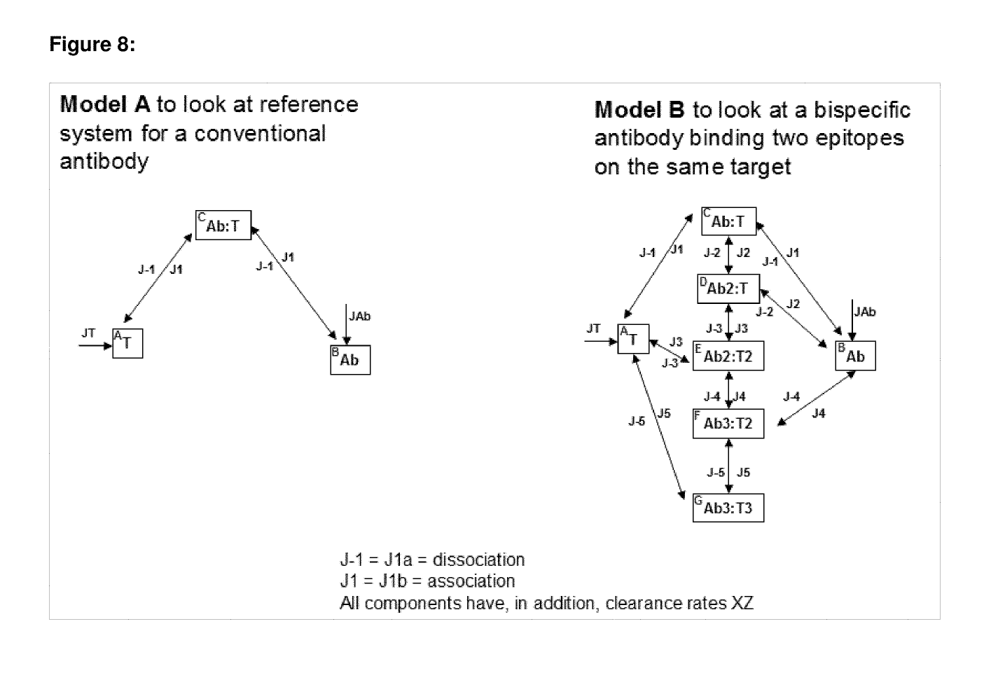

In order to study which effects bispecific antibodies of the invention have on the concentration of free soluble target, i.e. target that is available to exert its' biological potentially pathogenic action in patients, PKPD modeling was performed. The PKPD model that was used is illustrated as a graphical interaction model in FIG. 8. The parameters used in the model are given in Table 3 and reflect realistic generic parameters typical of therapeutic antibodies and cytokines.

TABLE 3
Parameters used in PKPD modeling

| Parameter | Value |
|---|---|
| KD(mAb) | 100 pM (kd = 1E−5 1/s; ka = 1E5 1/Ms) |
| Clearance rate mAb | 4E−7 1/s (T/2 20 days) |
| Clearance rate mAb complex | 8E−6 1/s (T/2 1 day) |
| containing 2 Fc or more | |
| Clearance rate target | 1E−3 1/s (T/2 12 min) |
| Production rate target | 1E−14 M/s (giving a level of 10 pM) |
| Dose | 1.2 mg/kg |
| Volume | 80 ml/kg |
| Dosing interval | Every 2 weeks |

### Effects of a Bispecific Antibody Compared to a Conventional Antibody Under Constant Target Production Rates:

In order to study which effects bispecific antibodies of the invention have on the concentration of free soluble target, i.e. target that is available to exert its' biological action, in cases where the target is produced at a constant rate, the following kinetic model was written in Berkeley Madonna.

### Model a for Conventional Antibodies:

Method RK4
Starttime=0
Stoptime=100*86400
DT=10
Time_days=time/86400
{rate equations}
d/dt(A)=JT+J1a−J1b−JAZ {cytokine}
d/dt(B)=JAb+J1a−J1b−JBZ {Antibody}
d/dt(C)=J1b−J1a−JCZ {Ab-cytokine complex}
Init A=1E-99
Init B=1E-99
Init C=1E-99
{flows}
JT=KT
JAb=Dose/ti*(time>t1)*(time<(t1+ti))+Dose/ti*(time>t2)*(time<(t2+ti))+
Dose/ti*(time>t3)*(time<(t3+ti))+Dose/ti*(time>t4)*(time<(t4+ti))+
Dose/ti*(time>t5)*(time<(t5+ti))+Dose/ti*(time>t6)*(time<(t6+ti))
J1a=C*CA
J1b=A*B*AC
JAZ=A*AZ
JBZ=B*BZ
JCZ=C*CZ
{Constants}
KT=1E-14 {cytokine production rate in moles/s}
Dose=1E-7 {Antibody dose in moles}
Ti=1200 {injection time in s}
T1=2*86400
T2=16*86400
T3=30*86400
T4=44*86400
T5=58*86400
T6=72*86400
CA=1E-5 {Ab dissociation rate constant in 1/s}
AC=1E5 {Ab association rate constant in moles/s}
AZ=1E-3 {T/2 11.5 min}
BZ=4E-7 {T/2 20 days}
CZ=4E-7 {T/2 20 days}

### Model B for Bispecific Antibodies of the Invention:

Method RK4
Starttime=0
Stoptime=100*86400
DT=10
Time_days=time/86400
{rate equations}
d/dt(A)=JT+J1a+J3a+J5a−J1b−J3b−J5b−JAZ {cytokine}
d/dt(B)=JAb+J1a+J2a+J4a−J1b−J2b−J4b−JBZ {Ab}
d/dt(C)=J1b+J2a−J1a−J2b−JCZ {AbCy}
d/dt(D)=J2b+J3a−J2a−J3b−JDZ {Ab2Cy}
d/dt(E)=J3b+J4a−J3a−J4b−JEZ {Ab2Cy2}
d/dt(F)=J4b+J5a−J4a−J5b−JFZ {Ab3Cy2}
d/dt(G)=J5b−J5a−JGZ {Ab3Cy3}
Init A=1E-99
Init B=1E-99
Init C=1E-99
Init D=1E-99
Init E=1E-99
Init F=1E-99
Init G=1E-99
{flows}
JT=KT
JAb=Dose/ti*(time>t1)*(time<(t1+ti))+Dose/ti*(time>t2)*(time<(t2+ti))+
Dose/ti*(time>t3)*(time<(t3+ti))+Dose/ti*(time>t4)*(time<(t4+ti))+
Dose/ti*(time>t5)*(time<(t5+ti))+Dose/ti*(time>t6)*(time<(t6+ti))
J1a=C*CA
J1b=A*B*AC
J2a=D*DB
J2b=B*C*BD
J3a=E*EA
J3b=D*A*AE
J4a=F*FB
J4b=B*E*BF
J5a=G*GA
J5b=A*F*AG
JAZ=A*AZ
JBZ=B*BZ
JCZ=C*CZ
JDZ=D*DZ
JEZ=E*EZ
JFZ=F*FZ
JGZ=G*GZ
{Constants}
KT=1E-14 {cytokine production rate in moles/s}
Dose=1E-7 {Antibody dose in moles}
Ti=1200 {injection time in s}
T1=2*86400
T2=16*86400
T3=30*86400
T4=44*86400
T5=58*86400
T6=72*86400
CA=1E-5 {Absite1 dissociation rate constant in 1/s}
AC=1E5 {Absite1 association rate constant in moles/s}
DB=CA {Absite2 dissociation rate constant in 1/s}
BD=AC {Absite2 association rate constant in moles/s}
EA=CA
AE=AC
FB=CA
BF=AC
GA=CA
AG=AC
AZ=1E-3 {T/2 11.5 min}
BZ=4E-7 {T/2 20 days}
CZ=BZ
DZ=8E-6 {T/2 1 day1}
EZ=DZ
FZ=DZ
GZ=DZ

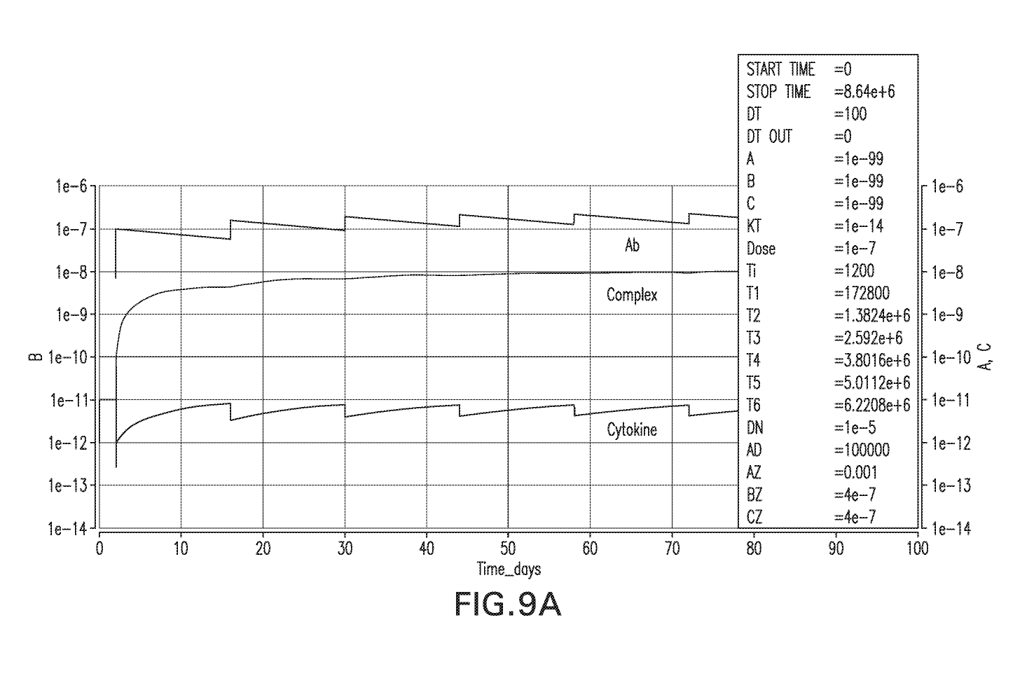

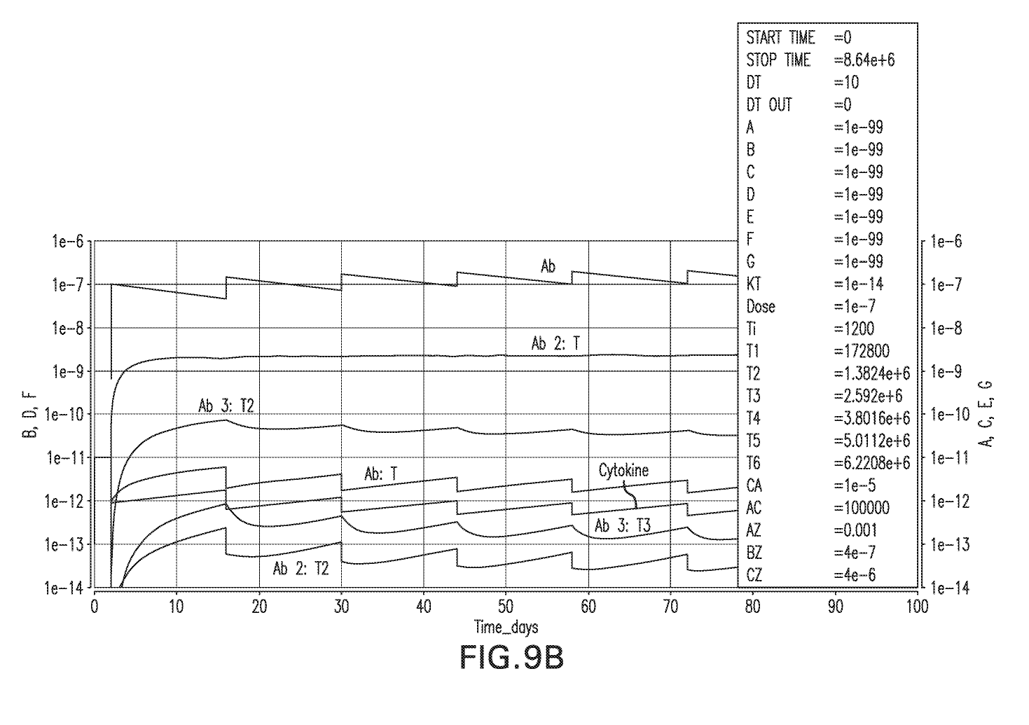

The models were run using the software Berkeley Madonna (Berkeley Madonna Inc., CA) to generate concentration curves of free soluble target. The result from model A (conventional antibody) is shown in FIG. 9A and the result from model B (bispecific antibody of the invention) is shown in FIG. 9B. It is important to note the logarithmic y-scale reflecting the concentration of free monomeric target. As can be seen from FIG. 9A, the level of free cytokine is only slightly repressed below the normal level of 10 pM when using a conventional antibody whereas, surprisingly, it is strongly repressed to below 1 pM when a bispecific antibody of the invention is used. This result is obtained comparing two antibodies (the conventional and the bispecific) which only differ in the bispecific antibodies ability to bind two different non-overlapping blocking epitopes on the target but on all other parameters such as KD, PK, dose of binding site and dosing interval are identical. The result shows that using a bispecific antibody of the invention, far superior blocking of biologically active soluble target can be achieved in vivo compared to a conventional monospecific antibody.

### Effects of a Bispecific Antibody Compared to a Conventional Antibody Under Non-Constant Target Production Rates

In order to study the influence of non-constant target production rates the target production was varied over time to simulate bursts in target production. Such bursts may be highly relevant for e.g. inflammatory cytokines where they are reported to occur during exacerbation of autoimmune disease. Both models A and B were modified to include a term for the variable production rate of the target:
JT=KT+AT
AT=BT*(exp(−WT*(time-tt1)̂2)+exp(−WT*(time-tt2)̂2)+exp(−WT*(time-tt3)̂2)+exp(−WT*(time-tt4)̂2)+exp(−WT*(time-tt5)̂2)+exp(−WT*(time-tt6)̂2)+exp(−WT*(time-tt7)̂2)+exp(−WT*(time-tt8)̂2)+exp(−WT*(time-tt9)̂2)+exp(−WT*(time-tt10)̂2)+exp(−WT*(time-tt11)̂2)+exp(−WT*(time-tt12)̂2)+exp(−WT*(time-tt13)̂2)+exp(−WT*(time-tt14)̂2)+exp(−WT*(time-tt15)̂2))
BT=2E-12 {peak cytokine production rate}
WT=5e-9 {with of target peak}

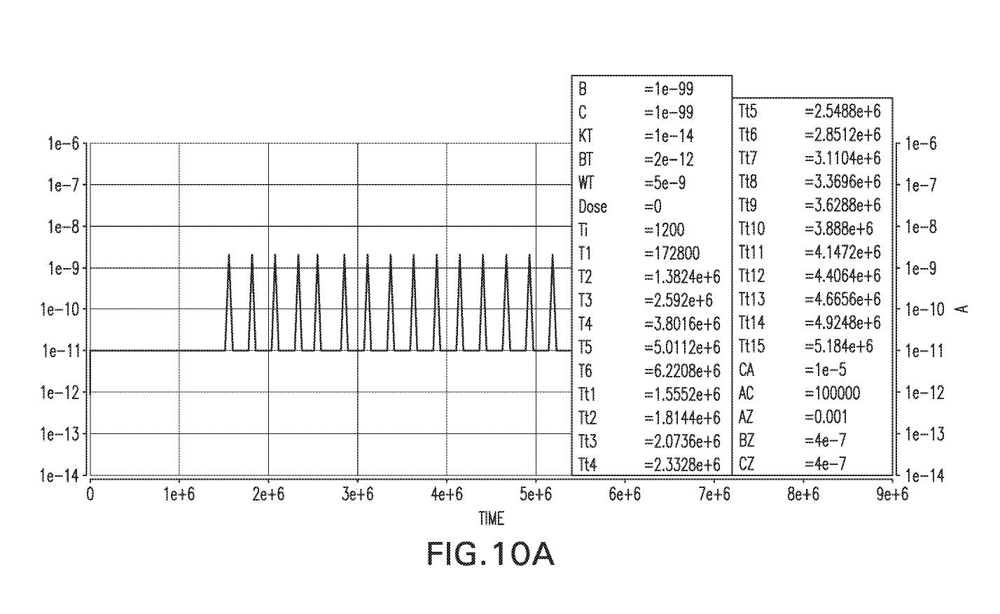

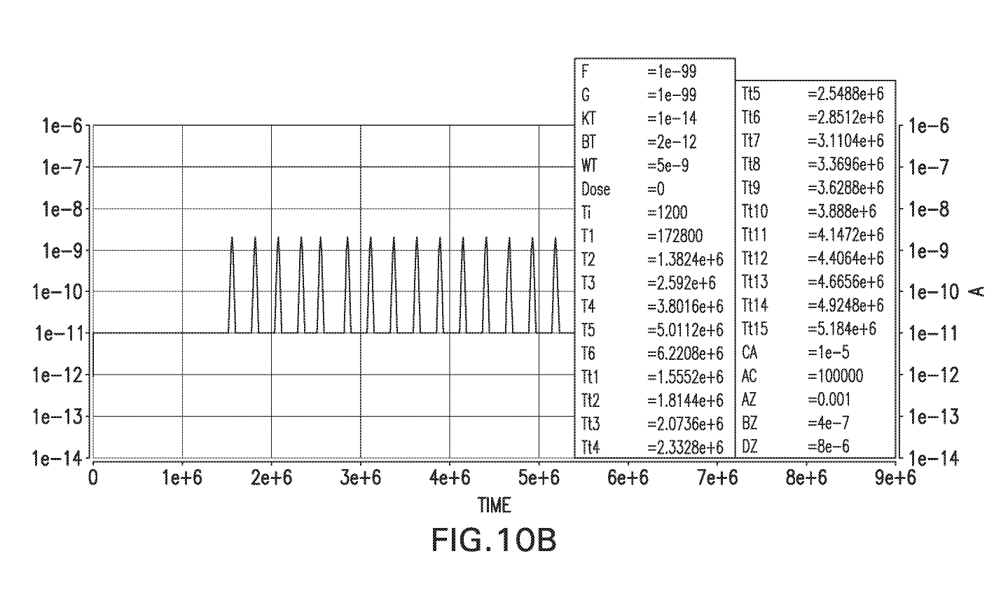

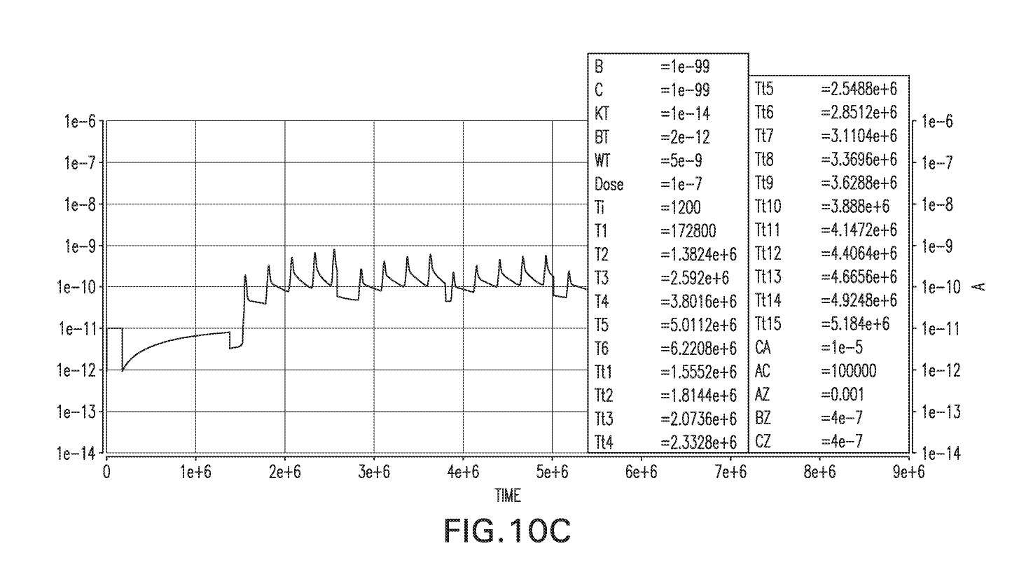

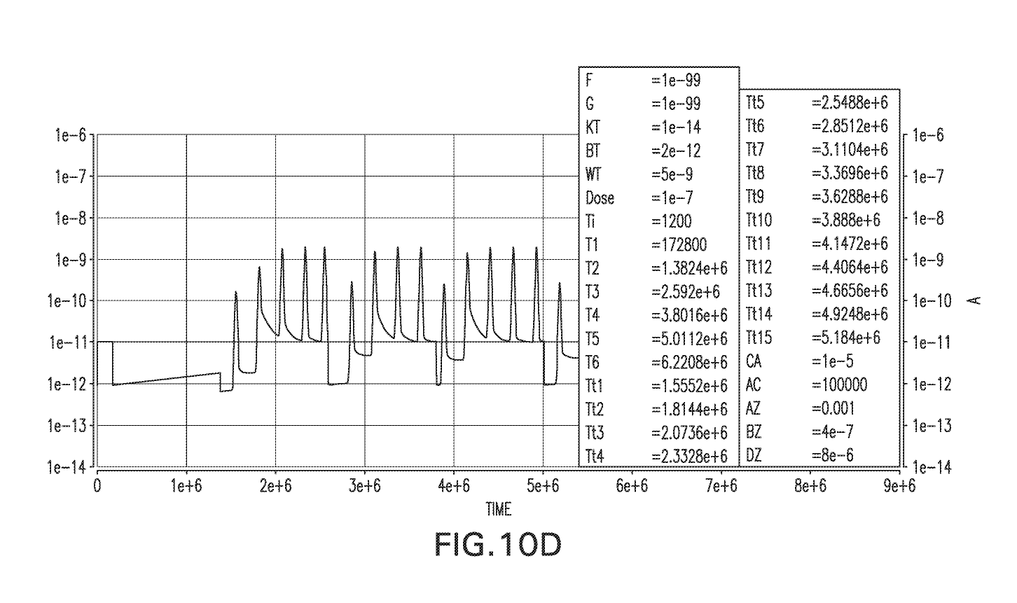

A run of this modified method without antibody is shown in FIGS. 10A and 10B, and the run for model A and model B with antibody is shown in FIGS. 10C and 10D, respectively. Again it is important to note the logarithmic y-scale reflecting the concentration of free monomeric target. As can be seen by comparing FIGS. 10A and 10C, surprisingly, a conventional monospecific antibody leads to a type of memory effect resulting in higher concentrations of free target between the target production bursts. This may in fact lead to more pronounced biological effects of the monomeric target when the conventional monospecific antibody is present compared to the situation without antibody. Crucially, a bispecific antibody of the invention shows a much diminished memory effect, leading to far less biologically active free monomeric target, as can be seen by comparing FIG. 10B with FIG. 10D.

### Example 9: PK Study

### ¹²⁵I-IL6 PK Study in Mice

To investigate and compare the clearance of IL6 by conventional monospecific antibodies and bispecific antibodies according to the invention a PK and tissue distribution study is performed in mice. Suitable antibodies according to the invention used in this context include antibodies of murine IgG2a isotype. Comment RB to BV: Please remove the following sentence if it is not necessary: One suitable antibody according to the invention used in this context would be antibody DVD-45 as shown in Table 1, however comprising a murine IgG2a rather than a human IgG1 Fc region.

### Clearance of Pre-Formed Complex

IL6 radio-labeled with 125I is obtained from a commercial source (Perkin Elmer Life and Analytical Sciences, Waltham, Mass.) and mixed with non-labeled IL6 and antibody so that the molar concentration of antibody binding sites about equals the molar concentration of IL6 epitopes (cold+labeled). The mixture is administered by intravenous injection into mice at an amount of about 1 mg antibody/kg body weight. IL6 without antibody is included as reference. At 0.083, 0.25, 0.5, 1, 2, 4, 6, 8, 24, 48, 96, and 192 hours, groups of 3 mice are sacrificed, blood plasma and organ samples prepared, and the protein-associated radioactivity measured using a gamma-counter. Relevant organs include kidney, liver and muscle. Further, urine samples are collected after every 24 hours.

In this theoretical example, a conventional monospecific anti-IL6 antibody increases the area under the curve (AUC) of the IL6 plasma-time curve at least 20-fold relative to IL6 without antibody, whereas a bispecific antibody of the invention decreases the IL6 AUC at least 3-fold relative to a conventional monospecific anti-IL6 antibody. Further, due to the clearance pathway of immune-complexes between IL6 and bispecific antibodies of the invention, the AUC of the IL6 liver-time curve is increased at least 3-fold for the bispecific antibodies of the invention relative to IL6 alone.

Clearance from Mice Pre-Treated with Antibody

IL6 radio-labeled with 125I is obtained from a commercial source (Perkin Elmer Life and Analytical Sciences, Waltham, Mass.) and mixed with non-labeled IL6. About 1 microgram/mouse of this mixture is administered by intravenous injection into mice, which have been pre-treated with about 5 microgram antibody/mouse about 6 h previously. The IL6 dose corresponds to the higher range of amounts of IL6 observed in patients with multiple myeloma or in animals exposed to bacterial infection. At 0.083, 0.25, 0.5, 1, 2, 4, 6, 8, 24, 48, 96, and 192 h, groups of 3 mice are sacrificed, blood plasma and organ samples prepared, and the protein-associated radioactivity measured using a gamma-counter. Relevant organs include kidney, liver and muscle. Further, urine samples are collected after every 24 hours.

In this theoretical example, a conventional monoclonal IL6 antibody increases the area under the curve (AUC) of the IL6 plasma-time curve at least 20-fold relative to IL6 without antibody, and a bispecific antibody of the invention decreases the IL6 AUC at least 3-fold relative to a conventional monospecific anti-IL6 antibody. Further, due to the clearance pathway of immune-complexes between IL6 and bispecific antibodies of the invention, the AUC of the IL6 liver-time curve is increased at least 3-fold for the bispecific antibodies of the invention relative to IL6 alone.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

To the extent possible under the respective patent law, all patents, applications, publications, test methods, literature, and other materials cited herein are hereby incorporated by reference.

What is claimed is:

1-26. (canceled)

27. A method for removing a soluble monomeric biomolecule from a bodily fluid by the formation of multimeric complexes using a binding molecule comprising at least two different binding sites, wherein at least one binding site is specific for an epitope present on said biomolecule, comprising the step of: contacting said bodily fluid with said bispecific binding molecule.

28. The method of claim 27, wherein said binding molecule comprises at least a first and a second binding site with specificity for two different epitopes on said monomeric biomolecule.

29. The method of claim 28, wherein the two different epitopes on said monomeric biomolecule do not overlap.

30. The method of claim 27, wherein said bispecific binding molecule comprises a first binding site with specificity for a first epitope on said soluble monomeric biomolecule, and a second binding site with specificity for a second epitope on a second soluble biomolecule present in said bodily fluid, wherein said second biomolecule comprises at least two copies of said second epitope.

31. The method of claim 27, wherein said bispecific binding molecule comprises at least two independent paratopes, wherein the first paratope is able to specifically bind a first epitope present on said monomeric soluble target molecule and the second paratope is able to specifically bind a second epitope present on a multimeric soluble target molecule.

32. The method of claim 31, wherein said bispecific binding molecule is able to bind said monomeric target biomolecule and said multimeric target molecule simultaneously.

33. The method of claim 31, wherein the monomeric soluble target biomolecule and the multimeric soluble target molecule are both human cytokines.

34. The method of claim 33, wherein the monomeric soluble target biomolecule is human GM-CSF and the multimeric soluble target molecule is human TNF-alpha.

35. The method of claim 33, wherein the monomeric soluble target biomolecule is human IL-6 and the multimeric soluble target molecule is human TNF-alpha.

36. The method of claim 33, wherein the monomeric soluble target biomolecule is human IL-6 and the multimeric soluble target molecule is human VEGF165.

37. The method of claim 27, wherein said binding molecule is a bispecific antibody molecule.

38. The method of claim 27, said binding molecule comprises an Fc region.

39. The method of claim 38, wherein said binding molecule comprises a human IgG1 Fc region.
